(19) United States
(12) Patent Application Publication
(10) Pub. No.: US 2010/0016300 A1
(43) Pub. Date: Jan. 21, 2010

(54) Imidazoacridine Compounds for Treating FLT3-Mediated Disorders

(76) Inventor: Alfred M. Ajami, Brookline, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/375,997

(22) PCT Filed: Aug. 2, 2007
(86) PCT No.: PCT/US07/17230

§ 371 Date: May 14, 2009

Related U.S. Application Data
(60) Provisional application No. 60/835,028, filed on Aug. 2, 2006.

(51) Int. Cl. A61K 31/5377; A61K 31/439; A61P 37/00

(52) U.S. Cl. 514/233.2; 514/281

(57) ABSTRACT

A method of treating a FLT3-mediated condition in a patient in need thereof, comprising administering to the patient a therapeutically effective amount of a compound of formula (I) or a pharmaceutically acceptable salt thereof: (I), The conditions that can be treated by the compounds of the present invention and the definitions for the variables in formula (I) are provided herein.

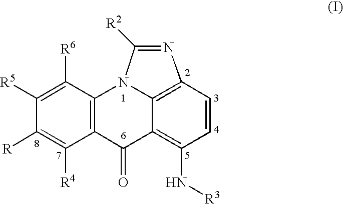

### RELATED APPLICATION

This application claims the benefit of U.S. Provisional Application 60/835,028, filed on Aug. 2, 2006. The entire teachings of the above application is incorporated herein by reference.

### BACKGROUND OF THE INVENTION

There is a need for a pharmaceutically acceptable therapies for inflammatory, immunocompromising, degenerative and demyelinating disorders, including the ones mediated by the FLT3 protein kinase, with high clinical efficacy that provides long-lasting clinical benefit without significant side effects.

### SUMMARY OF THE INVENTION

The present invention is a method of treatment of certain FLT3-mediated immune and autoimmune disorders by administration of certain derivatives of imidazoacridines.

In one embodiment, the present invention is a method of treating a FLT3-mediated condition in a patient in need thereof, comprising administering to the patient a therapeutically effective amount of a compound of formula (I) or a pharmaceutically acceptable salt thereof. The compounds used in the present invention are described by formula (I) below or a pharmaceutically acceptable salt thereof.

In formula (I),

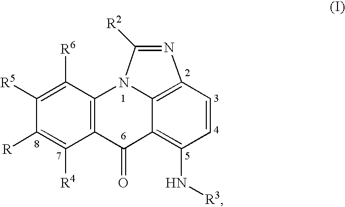

R is R^{x}, or R and R⁴ or, alternatively, R and R⁵ taken together with their intervening carbon atoms form a 5, 6 or 7 member, optionally substituted, cycloalkyl or non-aromatic heterocycle containing one or two oxygens and optionally substituted with methyl or hydroxyl; or

R is a hydrolyzable group; or

R, alone or taken together with R⁴, or alternatively R⁵, and their intervening carbon atoms is a phenol isosteric group.

R^{x} is —H, an optionally substituted alkyl, hydroxyl, alkoxy group, a halogen, or a group represented by the following structural formula:

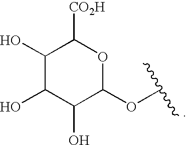

R² is —H, an optionally substituted C1-C10 alkyl or an optionally substituted aryl, optionally substituted aralkyl or optionally substituted heteroaryl.

R³ is —(CH₂)ₙ—NR^{a}R^{b}, wherein n is an integer from 1 to 5, and R^{a} and R^{b}, each independently are hydrogen or an optionally substituted alkyl, or R^{a} and R^{b}, taken together with the nitrogen to which they are attached, form group R^{y}. R^{y} is a heteroaryl or a non-aromatic heterocycle, each optionally substituted at one or more substitutable carbon atoms with methyl, hydroxyl, or methoxy, and optionally substituted at any substitutable ring nitrogen atom with C1-C4 alkyl or C1-C4 alkyl substituted with —NR^{c}R^{d}, wherein R^{c} and R^{d} are individually H, methyl or ethyl.

R⁴, R⁵ and R⁶, are each independently —H, —OH, a halogen or optionally substituted C1-C6 alkoxy; or R⁵ and R⁶ taken together with their intervening carbon atoms, form a 5, 6 or 7 member, optionally substituted cycloalkyl or optionally substituted non-aromatic heterocycle.

FLT3-mediated conditions that can be treated according to the methods of the present invention include axonal degeneration, acute transverse myelitis, amyotrophic lateral sclerosis, infantile spinal muscular atrophy, juvenile spinal muscular atrophy, Creutzfeldt-Jakob disease, subacute sclerosing panencephalitis, organ rejection, bone marrow transplant rejection, non-myeloablative bone marrow transplant rejection, ankylosing spondylitis, aplastic anemia, Behcet's disease, graft-versus-host disease, Graves' disease, autoimmune hemolytic anemia, Wegener's granulomatosis, hyper IgE syndrome, idiopathic thrombocytopenia purpura, or Myasthenia gravis.

### BRIEF DESCRIPTION OF THE DRAWINGS

FIG. 1 shows kinase screening phases.

FIG. 2 shows examples of novel FLT3 inhibitors conforming to Formula (I).

FIG. 3 shows FLT3 inhibitor activities of the compound depicted in FIG. 2.

FIG. 4A is a table that shows a detailed analysis of the FLT3 inhibitor selectivity of a number of known inhibitors in current development or recently approved as therapeutic drugs.

FIG. 4B is a table that illustrates IC50 values of compounds XF-22 and XF-113 against a representative selection of kinases.

FIG. 5 shows the plots of cell viability (left panel—RS4 cells; right panel—MV4 cells) as a function of effective concentration of the FLT3 inhibitors shown in FIG. 2.

FIG. 6 shows graphically that the mice treated with XF-2 (FLT3) inhibitor have lower clinical scores indicative of disease amelioration

### DETAILED DESCRIPTION OF THE INVENTION

It has now been discovered that administration of certain imidazoacridines can be used to treat certain FLT3-mediated conditions in a patient.

Specifically, it has been discovered that such FLT3-mediated inflammatory, immune or autoimmune disorders such as axonal degeneration, acute transverse myelitis, amyotrophic lateral sclerosis, infantile spinal muscular atrophy, juvenile spinal muscular atrophy, Creutzfeldt-Jakob disease, subacute sclerosing panencephalitis, organ rejection, bone marrow transplant rejection, non-myeloablative bone marrow transplant rejection, ankylosing spondylitis, aplastic anemia, Behcet's disease, graft-versus-host disease, Graves' disease, autoimmune hemolytic anemia, Wegener's granulomatosis, hyper IgE syndrome, idiopathic thrombocytopenia purpura, and Myasthenia gravis can be treated or alleviated by administering to a patient suffering from such a disease a therapeutically effective amount of a compound of formula (I) shown below or pharmaceutically acceptable salts thereof.

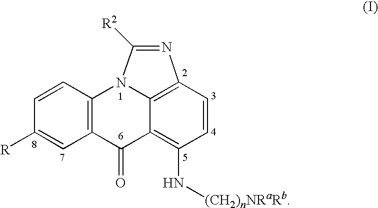

Values and preferred values for the variables in formula (I) are provided in the following paragraphs.

In one embodiment, R is either R^{x} or taken together with R⁴ or, alternatively, R⁵ and their intervening carbon atoms form a 5, 6 or 7 member, optionally substituted, cycloalkyl or non-aromatic heterocycle containing one or two oxygens and optionally substituted with methyl or hydroxyl. Preferably, R is R^{x}, wherein R^{x} is —H, an optionally substituted alkyl, hydroxyl, alkoxy group, a halogen. More preferably, R^{x} is —F, —OH or —OCH₃.

In another embodiment, R is a hydrolysable group. Preferably, R is selected from groups (II)-(VII):

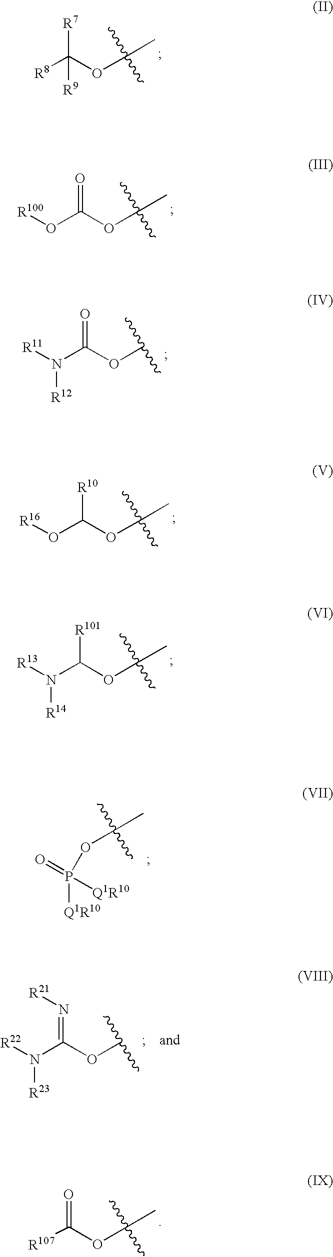

In another embodiment, R, alone or taken together with R⁴, or alternatively R⁵, and their intervening carbon atoms is a phenol isosteric group. Preferably, the phenol isosteric group is selected from:

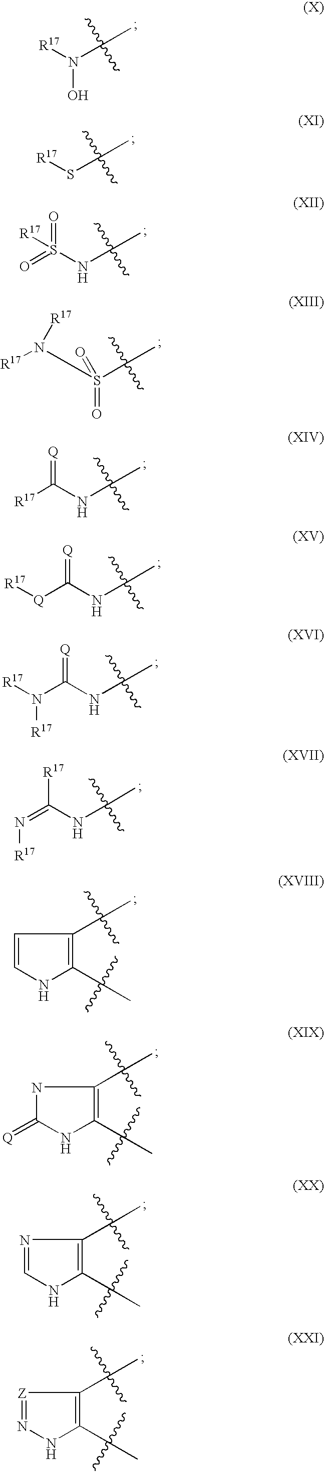

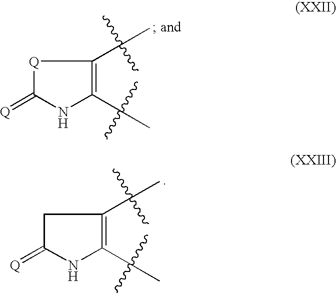

R² is —H, an optionally substituted C1-C10 alkyl or an optionally substituted aryl, optionally substituted aralkyl or optionally substituted heteroaryl. Preferably, R² is an optionally substituted C1-C10 alky. More preferably, R² is —H, C1-C4 alkyl or C1-C4 haloalkyl. Even more preferably, R²—H, methyl or ethyl.

R³ is —(CH₂)ₙ—NRₐR^{b}, wherein n is an integer from 1 to 5, and R^{a} and R^{b}, each independently are hydrogen or an optionally substituted alkyl, or R^{a} and R^{b}, taken together with the nitrogen to which they are attached, form group R^{y}.

R^{y} is a heteroaryl or a non-aromatic heterocycle, each optionally substituted at one or more substitutable carbon atoms with methyl, hydroxyl, or methoxy, and optionally substituted at any substitutable ring nitrogen atom with C1-C4 alkyl or C1-C4 alkyl substituted with —NR^{c}R^{d}, wherein R^{c} and R^{d} are individually H, methyl or ethyl. Preferably, R^{y} is an optionally substituted heteroaryl. Alternatively, R^{y} selected from:

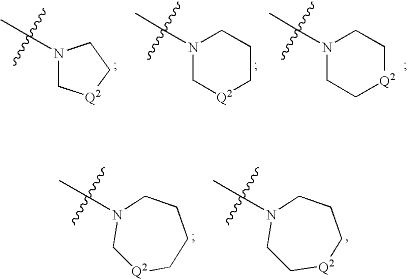

wherein Q² is S, O, CH₂, NH, or NR¹⁰², wherein R¹⁰² is methyl or ethyl.

Preferably, R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. More preferably, R^{a} and R^{b}, is each independently a hydrogen or a C1-C3 alkyl. Alternatively, R^{a} and R^{b} are independently each a —H or an C1-C4 alkyl, or, taken together with the nitrogen to which they are attached, form a 5-7 membered nonaromatic heterocycle, and wherein the C1-C4 alkyl is optionally substituted with a hydroxyl, an amino, a C1-C4 N-alkyl-amino or a C1-C4 N,N-dialkylamino group. Preferably, the substituents on R^{a} and R^{b} are independently hydroxyethyl, aminoethyl, N-alkylaminoethyl and N,N-dialkylaminoethyl. Yet more preferably, R^{a} and R^{b} are identical and are methyl or ethyl, or, taken together with the nitrogen atom to which they are attached, form a morpholino group.

R⁴, R⁵ and R⁶, are each independently —H, —OH, a halogen or an optionally substituted C1-C6 alkoxy, or R⁵ and R⁶ taken together with their intervening carbon atoms, form a 5, 6 or 7 member, optionally substituted cycloalkyl or non-aromatic heterocycle. Preferably, R⁴, R⁵ and R⁶ are each independently —H, —OH, C1-C4 alkyl or C1-C4 haloalkyl, or R⁵ and R⁶ taken together are methylenedioxy. More preferably, R⁴ is —H, and R⁵ and R⁶ are each independently —H, —OH, C1-C4 alkyl or C1-C4 haloalkyl, or taken together are methylenedioxy.

R⁷ and R⁸ are independently each H, optionally substituted C1-C6 alkyl, optionally substituted aryl or optionally substituted aralkyl. Preferably, R⁷ and R⁸ are independently each H, optionally substituted C1-C6 alkyl or phenyl or benzyl, each optionally substituted with one or more hydroxyl, C1-C3 alkoxy, amino, alkylamino, halogen, haloalkyl or haloalkoxy groups. More preferably, R⁷ and R⁸ are each independently H, methyl or ethyl.

R⁹ is carboxyl, carboxamide optionally N-substituted or N,N′-disubstituted with C1-C4 alkyl, C1-C6 alkanoyl, C1-C6 carbalkoxy, or optionally substituted aroyl. Preferably, R⁹ is carboxyl, carboxamide optionally N-substituted or N,N′-disubstituted with a C1-C4 alkyl, C1-C4 alkanoyl, or C1-C4 carbalkoxy. More preferably, R⁹ is a C1-C4 alkanoyl.

R¹⁰ is H, optionally substituted C1-C6 alkyl or optionally substituted aryl or optionally substituted aralkyl. Preferably, R¹⁰ (formula (V) and, independently, in formula (VII)) is H or C1-C4 alkyl, phenyl, or benzyl, each optionally substituted with one or more hydroxyl, C1-C3 alkoxy, amino, alkylamino, halogen, haloalkyl or haloalkoxy groups. More preferably, each R¹⁰ (formula (V), and, independently, formula (VII)) is independently an H, or C1-C4 alkyl.

R¹¹ and R¹² are independently each H, optionally substituted C1-C6 alkyl or, taken together with the atom to which they are attached, form an optionally substituted non-aromatic heterocycle. Preferably, R¹¹ and R¹² (formula (IV)) are independently each a H, methyl or ethyl or, taken together with the nitrogen atom to which they are attached form non-aromatic heterocycle, optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. More preferably, in formula (IV), NR¹¹R¹² is N-pyrrolidinyl, N-piperidinyl, N-morpholinyl, N-thiomorpholinyl or N-piperazinyl, optionally N′-substituted or N′,N′-disubstituted with C1-C4 alkyl or C1-C4 alkyl substituted with —NR^{c}R^{d}, wherein R^{c} and R^{d} are individually H, methyl or ethyl.

R¹³ and R¹⁴ are each independently H, optionally substituted C1-C6 alkyl, optionally substituted C1-C6 alkanoyl, or optionally substituted aroyl, or, taken together with the atom to which they are attached, form an optionally substituted heteroaryl or non-aromatic optionally substituted heterocycle.

R¹⁶ is optionally substituted C1-C6 alkyl, optionally substituted aryl or aralkyl, C1-C6 alkanoyl, or optionally substituted aroyl. Preferably, R¹⁶ (formula (V)) is a C1-C6 alkanoyl, optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)_{q}—C(O)OH, wherein q is an integer from 1 to 6. More preferably, R¹⁶ (formula (V)) is a branched C3-C6 alkanoyl.

R¹⁷ is H, C1-C6 alkyl, C1-C6 alkoxyalkyl, optionally substituted aryl or aralkyl or heteroaryl, optionally substituted aryloxy, aralkyloxy or heteroaryloxy, Q is O or S, and Z is CH or N. Preferably, R¹⁷ is H, optionally substituted C1-C6 alkyl, or C1-C6 alkoxyalkyl, or phenyl, benzyl, phenyloxy or benzyloxy each optionally substituted with halogen, —NO₂, —NH₂, —COOH, C1-C3 alkyl, C1-C3 carbalkoxy, C1-C3 a alkoxy group, C1-C3 haloalkyl or C1-C3 haloalkoxy. More preferably, R¹⁷ is H, C1-C4 alkyl, or phenyl, each optionally substituted with one or more halogen atoms, —NO₂, —NH₂, —COOH, C1-C3 alkyl, C1-C3 carbalkoxy, C1-C3 a alkoxy group, C1-C3 haloalkyl or C1-C3 haloalkoxy. Even more preferably, R¹⁷ is H, C1-C4 haloalkyl or phenyl, each optionally substituted with one or more halogen atoms or C1-C3 haloalkyls. Yet more preferably, R¹⁷ is H, trifluoromethyl or phenyl substituted with one or more trifluoromethyls.

R²¹ is optionally substituted C1-C10 alkyl, or an optionally substituted aryl or aralkyl or, R²¹ and R²² taken together with their intervening atoms form a 5-7 membered non-aromatic heterocycle. Preferably, either R²¹ is optionally substituted C1-C10 alkyl, phenyl, or benzyl, each optionally substituted with a halogen, —NO₂, —NH₂, —COOH, alkyl, C1-C3 carbalkoxy, C1-C3 alkoxy group, C1-C3 haloalkyl or C1-C3 haloalkoxy, or R²¹ and R²², taken together with their intervening atoms, form a 5 or 6 membered non-aromatic heterocycle.

R²² and R²³ are each independently —H, or a optionally substituted C1-C6 alkyl, provided that R²² and R²³ are not simultaneously hydrogens. Preferably, R²² and R²³ are each independently —H, or a C1-C3 alkyl.

R¹⁰⁰ is optionally substituted C1-C6 alkyl or optionally substituted aryl or optionally substituted aralkyl. Preferably, R¹⁰⁰ (formula (III)) is a C1-C4 alkyl.

R¹⁰¹ is H, optionally substituted C1-C6 alkyl or optionally substituted aryl or optionally substituted aralkyl. Preferably, R¹⁰¹ is H or C1-C4 alkyl. More preferably, R¹⁰¹ (formula (VI)) is H, methyl or ethyl.

R¹⁰⁷ is optionally substituted C1-C6 alkyl, optionally substituted aryl or aralkyl, or a non-aromatic heterocycle, optionally substituted at one or more substitutable carbon atoms with methyl, hydroxyl, or methoxy, and optionally N′-substituted at any substitutable nitrogen atom with C1-C4 alkyl or C1-C4 alkyl substituted with —NR^{c}R^{d}. Preferably, R¹⁰⁷ (formula (IX)) is C1-C6 alkyl optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH2)_{q}—C(O)OH. More preferably, R¹⁰⁷ (formula (IX)) is C1-C6 alkyl or C1-C6 carboxyalkyl.

Q¹ is O or NH.

Preferably, the group of formula (VI) is represented by structural formulas (VIa) or (VIb):

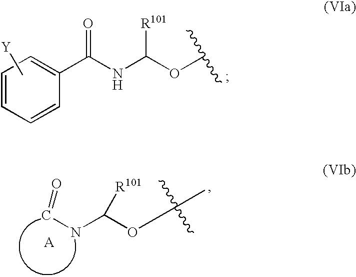

wherein Y is a halogen, —NO₂, —NH₂, —COOH, alkyl, C1-C3 carbalkoxy, C1-C3 alkoxy group, C1-C3 haloalkyl or C1-C3 haloalkoxy. Ring A is a 5-7 membered non-aromatic heterocycle optionally substituted at one or more substitutable ring carbon atoms with methyl, hydroxyl, oxy, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. Preferably, ring A in formula (VIb) is selected from:

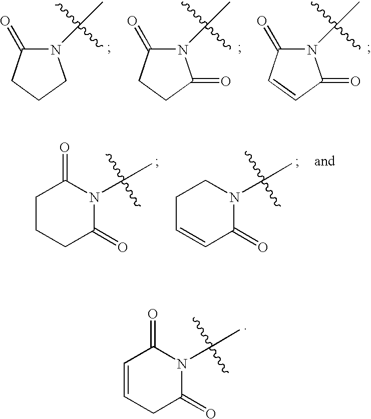

In one embodiment of the compound of formula (I), R is a hydrolyzable group selected from groups (II)-(VII); and R^{y} is an optionally substituted heteroaryl. Alternatively, R is a hydrolyzable group selected from groups (II)-(VII); and R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. Values and preferred values for the remainder of the variables are as described for formula (I).

In another embodiment, of the compound of formula (I), R is a hydrolyzable group selected from groups (II)-(VII); and

R^{y} is an optionally substituted heteroaryl or, alternatively, R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is a hydrolyzable group selected from groups (II)-(VII); and

n is 2 or 3;

R^{a} and R^{b} is each independently a hydrogen or a C1-C3 alkyl or R^{a} and R^{b}, taken together with the nitrogen to which they are attached, form group R^{y} selected form:

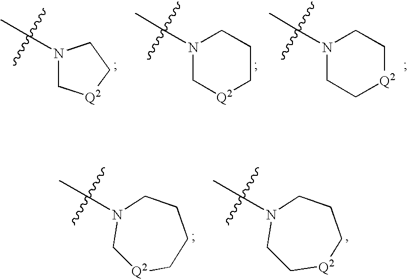

wherein Q² is S, O, CH₂, NH, or NR¹⁰², wherein R¹⁰² is methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is a hydrolyzable group selected from groups (II)-(VII); and

R^{y} is an optionally substituted heteroaryl or, alternatively, R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is a hydrolyzable group selected from groups (II)-(VII); and

n is 2 or 3;

R^{a} and R^{b} is each independently a hydrogen or a C1-C3 alkyl or R^{a} and R^{b}, taken together with the nitrogen to which they are attached, form group R^{y} selected form:

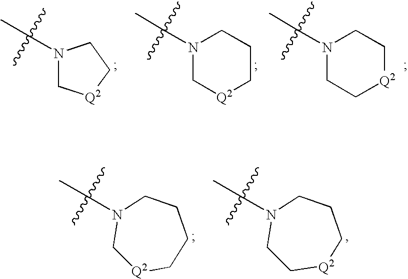

wherein Q² is S, O, CH₂, NH, or NR¹⁰², wherein R¹⁰² is methyl or ethyl; and

R²—H, methyl or ethyl. Values and preferred values for the remainder of the variables are as described for formula (I).

In one embodiment of the compound of formula (I), R is a phenol isosteric group selected from groups (X)-(XXIII); and R^{y} is an optionally substituted heteroaryl. Alternatively, R is a phenol isosteric group selected from groups (X)-(XXIII); and R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. Values and preferred values for the remainder of the variables are as described for formula (I).

In another embodiment, of the compound of formula (I), R is a phenol isosteric group selected from groups (X)-(XXIII)); and

R^{y} is an optionally substituted heteroaryl or, alternatively, R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is a phenol isosteric group selected from groups (X)-(XXIII); and

n is 2 or 3;

R^{a} and R^{b} is each independently a hydrogen or a C1-C3 alkyl or R^{a} and R^{b}, taken together with the nitrogen to which they are attached, form group R^{y} selected form:

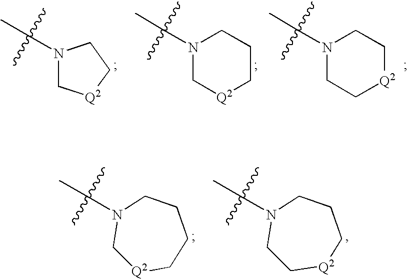

wherein Q² is S, O, CH₂, NH, or NR¹⁰², wherein R¹⁰² is methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is a phenol isosteric group selected from groups (X)-(XXIII); and

R^{y} is an optionally substituted heteroaryl or, alternatively, R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is a phenol isosteric group selected from groups (X)-(XXIII); and

n is 2 or 3;

R^{a} and R^{b} is each independently a hydrogen or a C1-C3 alkyl or R^{a} and R^{b}, taken together with the nitrogen to which they are attached, form group R^{y} selected form:

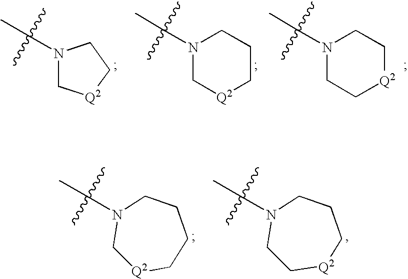

wherein Q² is S, O, CH₂, NH, or NR¹⁰², wherein R¹⁰² is methyl or ethyl; and

R²—H , methyl or ethyl. Values and preferred values for the remainder of the variables are as described for formula (I).

In one embodiment of the compound of formula (I), R is R^{x}; and R^{y} is an optionally substituted heteroaryl. Alternatively, R is R^{x}; and R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl. Values and preferred values for the remainder of the variables are as described for formula (I).

In another embodiment, of the compound of formula (I), R is R^{x}; and

R^{y} is an optionally substituted heteroaryl or, alternatively, R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is R^{x}; and

n is 2 or 3;

R^{a} and R^{b} is each independently a hydrogen or a C1-C3 alkyl or R^{a} and R^{b}, taken together with the nitrogen to which they are attached, form group R^{y} selected form:

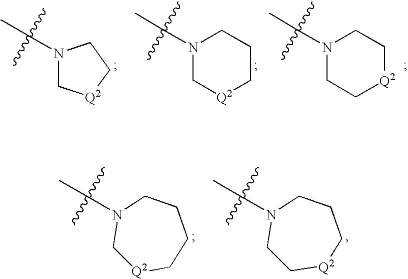

wherein Q² is S, O, CH₂, NH, or NR¹⁰², wherein R¹⁰² is methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is R^{x}; and

R^{y} is an optionally substituted heteroaryl or, alternatively, R^{a} and R^{b}, each independently are hydrogen or an alkyl, or R^{a} and R^{b}, taken together with the nitrogen to which they are attached, form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and

R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy. Values and preferred values for the remainder of the variables are as described for formula (I).

In another embodiment, of the compound of formula (I), R is R^{x}; and

n is 2 or 3;

R^{a} and R^{b} is each independently a hydrogen or a C1-C3 alkyl or R^{a} and R^{b}, taken together with the nitrogen to which they are attached, form group R^{y} selected form:

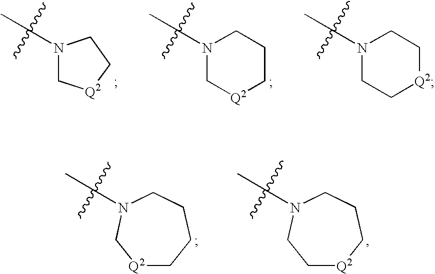

wherein Q² is S, O, CH₂, NH, or NR¹⁰², wherein R¹⁰² is methyl or ethyl; and

R²—H, methyl or ethyl. Values and preferred values for the remainder of the variables are as described for formula (I).

In one preferred embodiment, the compound of formula (I) is represented by structural formula (XXX):

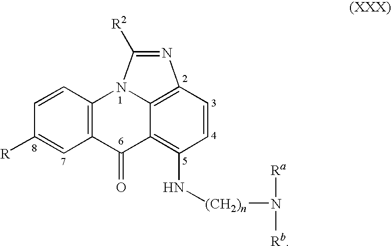

Values and preferred values for the variables of formula (XXX) are as described above for formula (I).

Preferably for formula (XXX), R is —F, —OH or —OCH₃ and the remainder of the variables take the values and preferred values defined above in formula (I).

More preferably, R is —F, —OH or —OCH₃, n is 2 or 3, and values and preferred values for R², R^{a} and R^{b} are as defined for formula (I).

Even more preferably, R is —F, —OH or —OCH₃, n is 2 or 3, and R²—H, methyl or ethyl. Values and preferred values for R^{a} and R^{b} are as defined for formula (I).

Yet more preferably, R is —F, —OH or —OCH₃, n is 2 or 3, and X is ═O, ═NH, or ═NOH, and R^{a} and R^{b} are each independently a C1-C3 alkyl. Alternatively, R is —F, —OH or —OCH₃, n is 2 or 3, R²—H, methyl or ethyl, and R^{a} and R^{b} are independently each a —H or an alkyl optionally substituted with a C1-C4 hydroxyalkyl.

In one preferred embodiment, R is —F, —OH or —OCH₃, R^{a} and R^{b} are identical and are methyl or ethyl, or taken together with the nitrogen atom to which they are attached, form a morpholino ring; n is 2 or 3; R²—H, methyl or ethyl. The remainder of the variables take the values and preferred values defined above in formula (I).

Examples of compounds of formula (I) include:

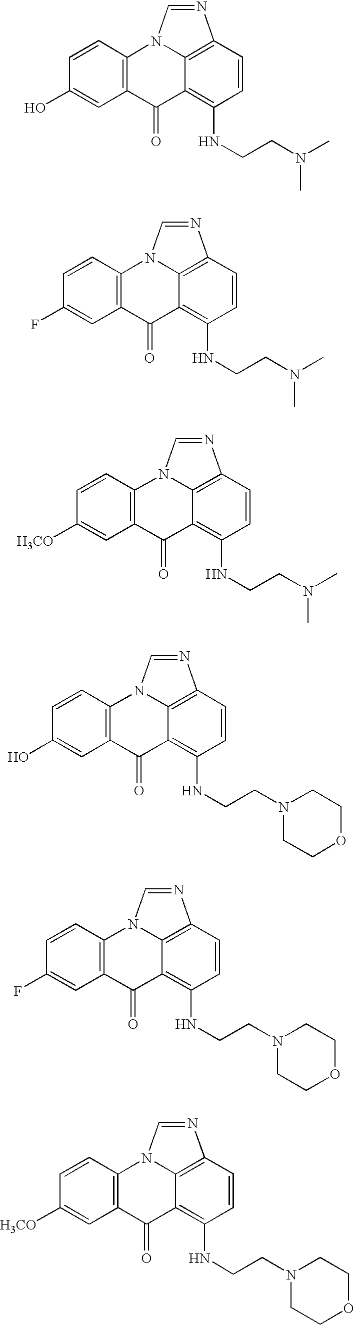

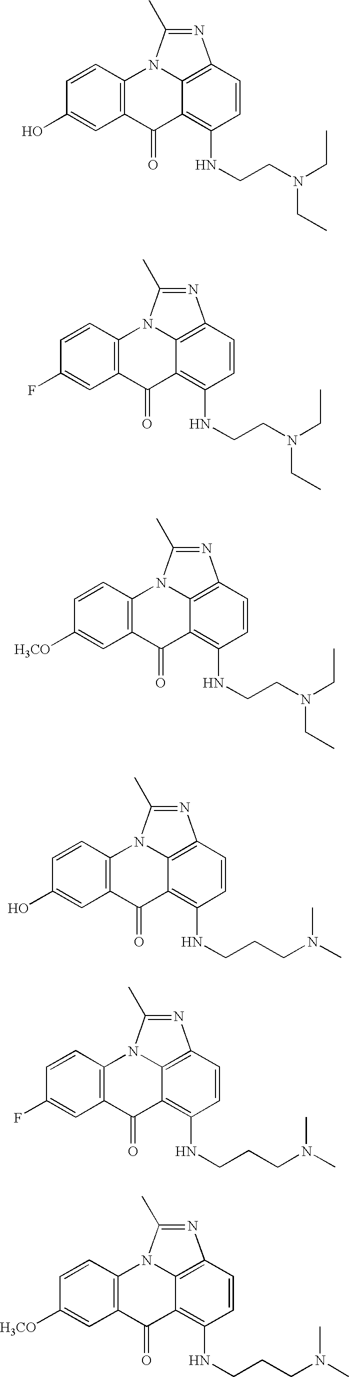

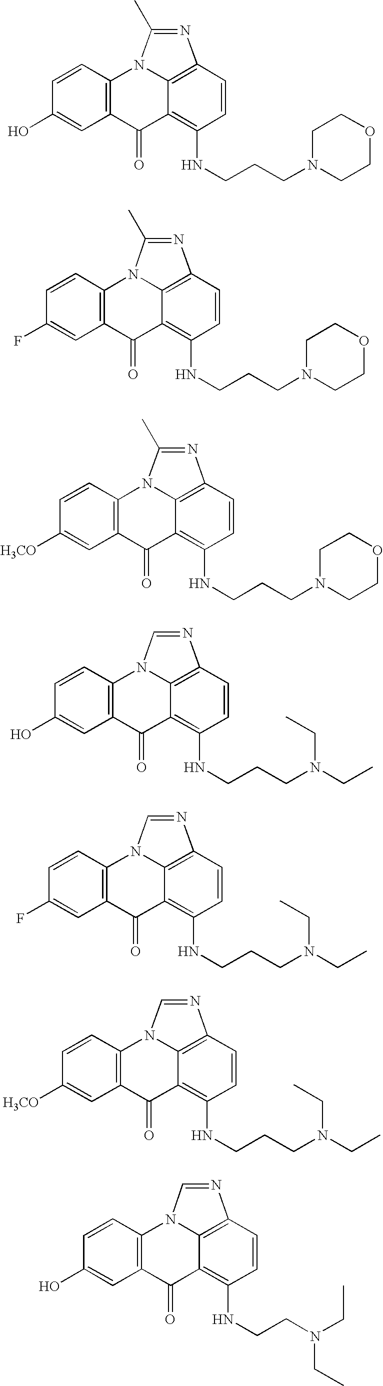

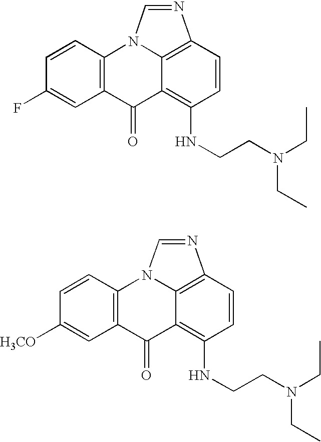

As used herein, the term “hydrolysable group” means a group which is hydrolyzed under physiological condition either spontaneously or by enzyme, e.g. esterase, peptidases, hydrolases, oxidases, dehydrogenases, lyases or ligases.

As used herein, the term “phenol isosteric group” means a chemical moiety whose electrostatic charge distribution, polarizability, capacity to form hydrogen bonds, hydrophobicity, steric effect and/or other properties are such that the group has chemical properties similar to a phenol.

As used herein, the term “optionally substituted imine” means a product of a reaction of a primary or a secondary amine with a carbonyl moiety. “Amine” is defined below.

The term “alkyl”, as used herein, unless otherwise indicated, includes straight or branched saturated monovalent hydrocarbon radicals, typically C1-C10, preferably C1-C6. Examples of alkyl groups include, but are not limited to, methyl, ethyl, propyl, isopropyl, and t-butyl. Suitable substituents for a substituted alkyl include —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)ₚ—(CH₂)_{q}—C(O)OH, where p and q are independently an integer from 1 to 6.

The term “cycloalkyl”, as used herein, is a non-aromatic saturated carbocyclic moieties, typically C3-C8. Examples of cycloalkyl include, but are not limited to, cyclopropyl, cyclobutyl, cyclopentyl, cyclohexyl, and cycloheptyl. Suitable substituents for a cycloalkyl are defined above for an alkyl.

The term “haloalkyl”, as used herein, includes an alkyl substituted with one or more F, Cl, Br, or I, wherein alkyl is defined above.

The terms “alkoxy”, as used herein, means an “alkyl-O—” group, wherein alkyl, is defined above.

The term “alkanoyl”, as used herein, means an “alkyl-C(O)—” group, wherein alkyl is defined above.

The term “haloalkoxy”, as used herein, means “haloalkyl-O—”, wherein haloalkyl is defined above.

As used herein, an amino group may be a primary (—NH₂), secondary (—NHRₓ), or tertiary (—NRₓR_{y}), wherein Rₓ and R_{y} may be any of the optionally substituted alkyls. “Alkyl” is described above. Preferably, Rₓ and R_{y} are unsubstituted alkyl groups.

The term “aryl”, as used herein, refers to a carbocyclic aromatic group. Examples of aryl groups include, but are not limited to phenyl and naphthyl.

The term “aryloxy”, as used herein, means an “aryl-O—” group, wherein aryl is defined above.

The term “aroyl”, as used herein, means an “aryl-C(O)—” group, wherein aryl is defined above.

The term “heteroaryl”, as used herein, refers to aromatic groups containing one or more heteroatoms (O, S, or N). A heteroaryl group can be monocyclic or polycyclic, e.g. a monocyclic heteroaryl ring fused to one or more carbocyclic aromatic groups or other monocyclic heteroaryl groups. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The term “heteroaryloxy”, as used herein, means a “heteroaryl-O—” group, wherein heteroaryl is defined above.

The term “non-aromatic heterocycle” refers to non-aromatic carbocyclic ring systems typically having four to eight members, preferably five to six, in which one or more ring carbons, preferably one to four, are each replaced by a heteroatom such as N, O, or S. Non aromatic heteroccyles can be optionally unsaturated. Examples of non-aromatic heterocyclic rings include 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrorolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, and 1-pthalimidinyl.

The heteroaryl or non-aromatic heterocyclic groups may be C-attached or N-attached (where such is possible). For instance, a group derived from pyrrole may be pyrrol-1-yl (N-attached) or pyrrol-3-yl (C-attached).

Suitable substituents for an aryl, a heteroaryl, or a non-aromatic heterocyclic group are those that do not substantially interfere with the pharmaceutical activity of the disclosed compound. One or more substituents can be present, which can be identical or different. Examples of suitable substituents for a substitutable carbon atom in aryl, heteroaryl or a non-aromatic heterocyclic group include —OH, halogen (—F, —Cl, —Br, and —I), —R′, haloalkyl, —OR′, —CH₂R′, —CH₂OR′, —CH₂CH₂OR′, —CH₂OC(O)R′, —O—COR′, —COR′, —SR′, —SCH₂R′, —CH₂SR′, —SOR′, —SO₂R′, —CN, —NO₂, —COOH, —SO₃H, —NH₂, —NHR′, —N(R′)₂, —COOR′, —CH₂COOR′, —CH₂CH₂COOR′, —CHO, —CONH₂, —CONHR′, —CON(R′)₂, —NHCOR′, —NR′COR′, —NHCONH₂, —NHCONR′H, —NHCON(R′)₂, —NR′CONH₂, —NR′CONR′H, —NR′CON(R′)₂, —C(═NH)—NH₂, —C(═NH)—NHR′, —C(═NH)—N(R′)₂, —C(═NR′)—NH₂, —C(═NR′)—NHR′, —C(═NR′)—N(R′)₂, —NH—C(═NH)—NH₂, —NH—C(═NH)—NHR′, —NH—C(═NH)—N(R′)₂, —NH—C(═NR′)—NH₂, —NH—C(═NR′)—NHR′, —NH—C(═NR′)—N(R′)₂, —NR′H—C(═NH)—NH₂, —NR′—C(═NH)—NHR′, —NR′—C(═NH)—N(R′)₂, —NR′—C(═NR′)—NH₂, —NR′—C(═NR′)—NHR′, —NR′—C(═NR′)—N(R′)₂, —SO₂NH₂, —SO₂NHR′, —SO₂NR′₂, —SH, —SOₖR′ (k is 0, 1 or 2) and —NH—C(═NH)—NH₂. Each R′ is independently an alkyl group. Oxo (C══O) and thio (C══S) are also suitable substituents for a non-aromatic heterocycle.

Suitable substituents on the nitrogen of a non-aromatic heterocyclic group or a heteroaryl group include —R″, —N(R″)₂, —C(O)R″, —CO₂R″, —C(O)C(O)R″, —C(O)CH₂C(O)R″, —SO₂R″, —SO₂N(R″)₂, —C(═S)N(R″)₂, —C(═NH)—N(R″)₂, and —NR″SO₂R″. R″ is hydrogen, an alkyl or alkoxy group.

Compounds of formula (I) can be synthesized according to a variety of synthetic schemes disclosed in U.S. Pat. Nos. 5,231,100 and 6,229,015, incorporated herein by reference in their entirety. One example of such a scheme is shown below:

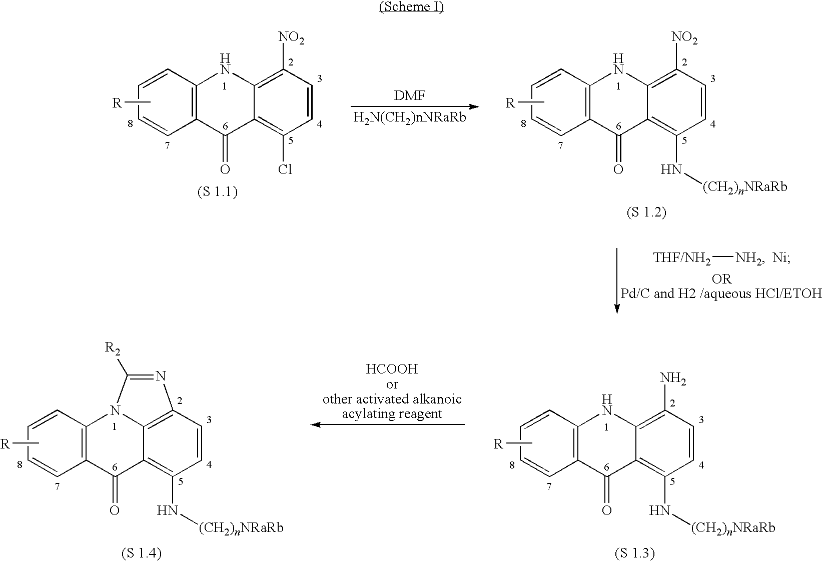

As used herein, the “activated alkanoic acylating agent” is defined within the references cited. For example: alkyl nitriles are reacted with HCl in methanol or ethanol to give the corresponding acetimidate ester hydrochlorides, R—CN going to R—C(OMe)═NH+Cl—, where R is an alkyl. This acetimidate reacts with the amine in compound (S 1.3) and cyclized to the methyl or ethyl or other alkylimidazole, again per the cited articles.

The methods of the present invention are directed to treating FLT3-mediated disorders, comprising administering to a subject a therapeutically effective amount of a compound disclosed herein, in particular compounds of formula (I) or a pharmaceutically acceptable salt thereof. Preferably, the compounds illustrated in the preceding examples are used.

The term “patient” means a warm blooded animal, such as for example rat, mice, dogs, cats, guinea pigs, and primates such as humans.

The terms “treat” or “treating” include any treatment, including, but not limited to, alleviating symptoms, eliminating the causation of the symptoms associated with a FLT-3-mediated condition either on a temporary or permanent basis, or preventing or slowing the appearance of symptoms and progression of the named disorder or condition.

As used herein the term “therapeutically effective amount” is the amount of a compound disclosed herein that will achieve a partial or total inhibition or delay of the progression of a FLT3-mediated disorder in a patient.

As used herein, the term “FLT3-mediated disorder” is a disorder in which one or more symptoms can be inhibited, alleviated, reduced or whose onset can be delayed by inhibiting completely or partially the FLT3 protein kinase.

As used herein, cross-inhibition or cross-reactivity, is defined as the property of not inhibiting other tyrosine kinases (this is a specific term of art that includes the receptor tyrosine kinases-RTKs and non-receptor tyrosine kinases-TK) at IC50 values within 100 fold of the IC50 against FLT3. For example, in the case of FX-2 (symadex) the IC50 is ˜10 against FLT3 and >2000 against FGFR2 and >5000 against all the other RTK's. Other so-called FLT3 inhibitors known in the prior art also inhibit other RTKs and TKs with IC50s less than 100 fold greater.

FLT3 (Fms-like tyrosine kinase; other names include CD135, FLK2 (Fetal liver kinase 2), STK1 (Stem cell kinase 1)) is a class III receptor tyrosine kinase (RTK) structurally related to the receptors for platelet derived growth factor (PDGF), colony stimulating factor 1 (CSF1), and KIT ligand (KL). These RTKs contain five immunoglobulin-like domains in the extracellular region and an intracelular tyrosine kinase domain split in two by a specific hydrophilic insertion (kinase insert). FLT3, closely related to PDGF receptors and c-Kit is, however, not inhibited by the small molecule inhibitors of PDGF and c-Kit; (G Del Zotto et al., J. Biol. Regulators Homeostatic Agents 15: 103-106, 2001)

FLT3 is reported to be highly expressed in brains, placentae, livers and hematopoietic stem cells (O. Rosnet et al., Blood, 82:1110-1119; A. Turner et al., Blood 88:3383-3390, 1996). The analysis of genes using knockout mice has revealed that the destruction of FLT3 genes leads to injury of precursor cells of lymphocytes. It is also reported that destruction of KIT genes simultaneously with the destruction of FLT3 genes causes severe hematopoietic injury involving pancytopenia (K. Mackarehtschian, Immunity, 3:147-161, 1995). Further, in knockout mice of FLT3 ligand, a reduction in leukocytic cells in bone marrow, bone marrow progenitor cells and B lymphoid progenitor cells, a deficit of natural killer cells in the spleen, and a reduction in dendritic cells of the spleen, thymus, and lymph nodes are observed (H. J. McKenna et al., Blood 95: 3489-3497, 2000).

Furthermore, in chronic myelocytic leukemia (CML), cases are reported in which, as compared with the chronic phase, the expression of FLT3 is increased after conversion to the acute phase (Iwai, T. et al., Leukemia, 11: 1992-1993. 1997). As described above, it is considered that, upon an enhancement in a signal transduction system as a result of occurrence of an abnormal phenomenon of FLT3, excessive growth and differentiation of haemopoietic cells take place, leading to tumorigenesis, immune disorder and the like of cells.

Activating mutations of the flt3 gene, located on 13q12.2, have been identified in a proportion of acute myelogenous leukemia (AML) patients. FLT3 is the most commonly mutated gene in AML, and is constitutively activated by acquired mutation in approximately 30%-35% of AML. In 20%-25% of cases of AML, there are internal tandem duplications (ITD) of a small number of amino acid residues in the juxtamembrane domain of FLT3, and in 10% there are activating mutations in other FLT3 domains (mainly in exon 14). These mutations activate the FLT3 kinase activity constitutively, and result in increased cellular proliferation and viability. AML patients with FLT3 mutations have a poor prognosis. This progress had led to the development of small molecules that specifically inhibit the abnormally activated FLT3 kinase.

Accordingly, in one embodiment, the present invention is a method of treating a patient suffering from an acute myeloid leukemia characterized by a FLT3 mutation. In one embodiment, the method comprises contacting a cell, selected from a list presented in the paragraphs below, in the patient with an effective amount of a compound of formula (I) or a pharmaceutically acceptable salt thereof.

A FLT3 mutation that causes AML or related myeloid or lymphocytic hematological malignancies can be identified by methods well-known in the art. These methods include polymerase chain reaction (PCR)-based amplification techniques, gene sequencing or differential gene expression with mRNA based microarrays. To date, for example, several primer pairs for the detection of FLT3 mutations have been described. They include, for example, the following primer sequences: for the detection of ITD mutations in the juxtamembrane domain: forward primer: 5-CAATTTAGGTATGAAAGCCAGC-3 (SEQ ID NO:1); reverse primer: 5-CTTTCAGCATTTTGACGGCAACC-3 (SEQ ID NO:2); for the detection of D835 mutations in the kinase domain: forward primer: 5-CCGCCAGGAACGTGCTTG-3 (SEQ ID NO:3); reverse primer: 5-GCAGCCTCACATTGCCCC-3 (SEQ ID NO:4). Detailed descriptions on methodology, incorporated herein by reference, are readily available in textbooks of molecular medicine (see, for example, H Kiyoi and T Naoe, Methods in Molecular Medicine, H. Iland et al., Eds, Humana Press, Chapter 12, pp. 189-197, 2005, the relevant portions of which are incorporated herein by reference). Specific sequencing and microarray methods that identify either point mutations directly or indirectly via informatic reconstruction of expressed sequence tags and other gene fragment constructs also have become routinely available for application to patient blood and tissue samples. Guidance on how these methodologies have been used to determine the status of FLT3 mutations is given by J Jiang et al. (Blood 104:1855-1858, 2004); M. Beran et al, (Leukemia Res. 28:547-550, 2004); and F. Kuchenbauer et al. (Hematologica 90:1617-1625, 2005). The entire teachings of these publications is incorporated herein by reference.

Various cell types express FLT3. Among the cells that express FLT3 are the following types: a dendritic cell, a NK cell, a T-cell, a B-cell, a glyal cell, an oligodendrocyte, a Schwann cell, an astrocyte, a mycroglia, afferent neuron, efferent neuron, interneuron, GABAergic neuron, cholinergic neuron, dopaminergic neuron, serotonergic neuron, neuroendocrine cell, postmitotic neuron, embryonic neuron, a ganglion cell in retina; B-cell progenitor cells, colony forming megakaryocytic (CFU-MK) cell, colony forming granulocytic-monocytic (CFU-GM) cell, colony forming granulocytic (CFU-G) cell, colony forming monocytic (CFU-M) cell, colony forming dendritic (CFU-D) cell, granulocyte-monocyte progenitor cell, Langerhans cell, lymphoblastic leukemia cell, lymphoma cell, monocyte, myeloid leukemia cell, natural killer (NK) progenitor cell, plasmacytoid dendritic cell, splenocyte, T-cell progenitor cell, and thymocyte.

The disclosed compounds are administered in a form or mode which makes the compound bioavailable in therapeutically effective amounts. For example, compounds of formula (I) can be administered in a form of a pharmaceutically acceptable salt. The term “pharmaceutically acceptable salts” means either an acid addition salt or a basic addition salt, whichever is possible to make with the compounds of the present invention. “Pharmaceutically acceptable acid addition salt” is any non-toxic organic or inorganic acid addition salt of the base compounds represented by formula (I) or the examples thereof. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points. For compounds comprising an acidic group “pharmaceutically acceptable basic addition salts” means non-toxic organic or inorganic basic addition salts of the compounds of formula (I), including the above-mentioned examples thereof. Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection criteria for the appropriate salt will be known to one skilled in the art.

Compounds used in the disclosed methods can be administered by a number of routes including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18^{th} Edition, Mack Publishing Co. (1990), incorporated herein by reference.

The compounds of formula (I), including the above-mentioned examples thereof, may also be administered topically, and when done so the carrier may suitably comprise a solution, ointment or gel base. The base, for example, may comprise one or more of petrolatum, lanolin, polyethylene glycols, bee wax, mineral oil, diluents such as water and alcohol, and emulsifiers and stabilizers.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The compounds used in the present invention can be administered alone or in combination with one or more other pharmaceutically active agents that are effective against the cancer being treated.

As used herein, the term “combination” with reference to pharmaceutically active agents and the term “co-administering” and “co-administration” refer to administering more than one pharmaceutically active agent to a patient during one treatment cycle and not necessarily simultaneous or in a mixture.

The dosage range at which the disclosed compounds of formula (I) exhibit their ability to act therapeutically can vary depending upon the severity of the condition, the patient, the formulation, other underlying disease states that the patient is suffering from, and other medications that may be concurrently administered to the patient. Generally, the inventive compounds of the invention will exhibit their therapeutic activities at dosages of between about 0.001 mg/kg of patient body weight/day to about 100 mg/kg of patient body weight/day. For example, the dosage can be 0.1-100 mg/kg, 1-100 mg/kg, 10-100 mg/kg, 1-50 mg, kg, 10-50 mg/kg or 10-30 mg/kg per day, per every other day or per week.

In other embodiments, compounds can be administered by any of the routes described below, preferably intravenously, in an amount from 1 mg per kilogram body weight to 20 mg per kg body weight. Compounds can be administered daily, once every 72 hours or weekly.

In one embodiment in which compounds are used to treat rheumatoid arthritis, compounds can be administered orally in an amount of 1-50 mg/kg, 10-40 mg/kg, 20-30 mg/kg or 30 mg per kilogram of body weight per day, per every other day or per week.

In one embodiment, the compounds of the invention are administered chronically to the patient in need thereof. For example, the chronic administration of the compound is daily, weekly, biweekly, or monthly over a period of at least one year, at least two years, at least three or more years.

In one embodiment, the compounds of formula (I) are administered intravenously in the amount of 1.5-30 mg/kg once at intervals of 1-3 months. In another embodiment, the compounds are administered orally in the amount of 5-100 mg/kg on same schedule as above. Alternatively, the compounds of formula (I) are administered several times over a period of up to 3 months and up to a cumulative dose of between 1.5 and 30 mg/kg. In another embodiment, the cumulative dose is from 5 to 100 mg/kg.

In another embodiment, the compounds of formula (I) are administered intravenously in the amount of 2.5-10 mg/kg weekly for 8-24 weeks, repeating as needed after 6-18 weeks off drug. Alternatively, the compounds of formula (I) are administered several times over a period of from 14 weeks to 42 weeks to achieve a cumulative dose from 20 mg/kg to 240 mg/kg. Administration can be repeated over one or more periods of 14-42 weeks.

In another embodiment, the compounds of formula (I) are administered intravenously in the amount of 2.5-10 mg/kg twice, 72 hrs apart for 1 to 2 weeks, repeating monthly. Alternatively, the compounds of formula (I) are administered several times over a period of up to two weeks, up to a cumulative dose of from 11 mg/kg to 47 mg/kg. Administration can be repeated monthly.

In another embodiment, the compounds of formula (I) are administered orally in the amount of 1-3 mg/kg daily for 10-15 days, repeating every 30-45 days. Alternatively, the compounds of formula (I) are administered several times over a period of up to 40-60 days, up to a cumulative dose of from 10 mg/kg to 45 mg/kg. Administration can be repeated over one or more periods of up to 40-60 days.

In another embodiment, the compounds of the invention are administered orally in the amount of 2-6 mg/kg daily for 3 days per week, repeating every 15-30 days. Alternatively, the compounds of formula (I) are administered several times over a period of up to 30 days up to a cumulative dose of 6-18 mg/kg. Administration can be repeated over one or more periods of up to 30 days.

Preferably, the administration of the compounds or the combinations of the compounds described herein results in an effective blood level of the compound in the patient of more than or equal to 10 ng/ml. For example, compounds can be administered intravenously in an amount of 20 μg to about 500 μg per kilogram body weight of the patient.

Preferred human doses for treating chronic (remitting-relapsing) multiple sclerosis (MS) are 0.1 mg/kg to 10 mg/kg, 1-10 mg/kg, 1-5 mg/kg, 2-7 mg/kg, 2-5 mg/kg. Schedule could be once a month, twice a month, three times a month or once or twice a week for 3 months, 6 month, 12 months or more.

Preferred human doses for treating acute MS, is 0.1 mg/kg to 10 mg/kg, 0.1-5 mg/kg, 0.1-2 mg/kg, 0.5-2 mg/kg or 0.5-1 mg/kg three times a day, twice a day, or daily, on a weekly, biweekly or monthly basis.

Preferred human doses for treating rheumatoid arthritis 0.1 mg/kg to 10 mg/kg, 1-10 mg/kg, 1-5 mg/kg, 2-7 mg/kg, 2-5 mg/kg three times a day, twice a day, or daily, on a weekly, biweekly or monthly basis.

In treating a patient afflicted with a condition described above, all of the disclosed compounds can be administered in any-form or mode which makes the compound bioavailable in therapeutically effective amounts. For example, compounds of formula (I) can be administered in a form of a pharmaceutically acceptable salt. The term “pharmaceutically acceptable salt” means either an acid addition salt or a basic addition salt, whichever is possible to make with the compounds of the present invention. “Pharmaceutically acceptable acid addition salt” is any non-toxic organic or inorganic acid addition salt of the base compounds represented by formula (I). Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicyclic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points. “Pharmaceutically acceptable basic addition salts” means non-toxic organic or inorganic basic addition salts of the compounds of formula (I). Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline. The selection of the appropriate salt may be important so that the ester is not hydrolyzed. The selection criteria for the appropriate salt will be known to one skilled in the art.

Compounds of the present invention can be administered by a number of routes including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18^{th} Edition, Mack Publishing Co. (1990), incorporated herein by reference.

The compound of formula (I) of this invention may also be administered topically, and when done so the carrier may suitably comprise a solution, ointment or gel base. The base, for example, may comprise one or more of petrolatum, lanolin, polyethylene glycols, bee wax, mineral oil, diluents such as water and alcohol, and emulsifiers and stabilizers.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The compounds used in the present invention can be administered alone or in combination with one or more other pharmaceutically active agents that are effective against the inflammatory condition and/or the demyelating disorder being treated.

As used herein, the term “combination” with reference to pharmaceutically active agents and the term “co-administering” and “co-administration” refer to administering more than one pharmaceutically active agent to a patient during one treatment cycle and not necessarily simultaneous or in a mixture.

When treating inflammatory conditions, for example acute transverse myelitis, organ rejection, bone marrow transplant rejection, non-myeloablative bone marrow transplant rejection, ankylosing spondylitis, Behcet's disease, graft-versus-host disease, Graves' disease, autoimmune hemolytic anemia, Wegener's granulomatosis, idiopathic thrombocytopenia purpura, and Myasthenia gravis, the compounds of the present invention are administered in combination with an anti-inflammatory agent. The anti-inflammatory agent can be adrenocorticotropic hormone, a corticosteroid, an interferon, glatiramer acetate, or a non-steroidal anti-inflammatory drug (NSAID).

Examples of suitable anti-inflammatory agents include corticosteroid such as prednisone, methylprednisolone, dexamethasone cortisol, cortisone, fludrocortisone, prednisolone, 6α-methylprednisolone, triamcinolone, or betamethasone.

Other examples of suitable anti-inflammatory agents include NSAIDs such as aminoarylcarboxylic acid derivatives (e.g., Enfenamic Acid, Etofenamate, Flufenamic Acid, Isonixin, Meclofenamic Acid, Niflumic Acid, Talniflumate, Terofenamate and Tolfenamic Acid), arylacetic acid derivatives (e.g., Acematicin, Alclofenac, Amfenac, Bufexamac, Caprofen, Cinmetacin, Clopirac, Diclofenac, Diclofenac Sodium, Etodolac, Felbinac, Fenclofenac, Fenclorac, Fenclozic Acid, Fenoprofen, Fentiazac, Flubiprofen, Glucametacin, Ibufenac, Ibuprofen, Indomethacin, Isofezolac, Isoxepac, Ketoprofen, Lonazolac, Metiazinic Acid, Naproxen, Oxametacine, Proglumrtacin, Sulindac, Tenidap, Tiramide, Tolectin, Tolmetin, Zomax and Zomepirac), arylbutyric acid Derivatives (e.g., Bumadizon, Butibufen, Fenbufen and Xenbucin) arylcarboxylic acids (e.g., Clidanac, Ketorolac and Tinoridine), arylproprionic acid derivatives (e.g., Alminoprofen, Benoxaprofen, Bucloxic Acid, Carprofen, Fenoprofen, Flunoxaprofen, Flurbiprofen, Ibuprofen, Ibuproxam, Indoprofen, Ketoprofen, Loxoprofen, Miroprofen, Naproxen, Oxaprozin, Piketoprofen, Piroprofen, Pranoprofen, Protinizinic Acid, Suprofen and Tiaprofenic Acid), pyrazoles (e.g., Difenamizole and Epirizole), pyrazolones (e.g., Apazone, Benzpiperylon, Feprazone, Mofebutazone, Morazone, Oxyphenbutazone, Phenylbutazone, Pipebuzone, Propyphenazone, Ramifenazone, Suxibuzone and Thiazolinobutazone), salicyclic acid derivatives (e.g., Acetaminosalol, 5-Aminosalicylic Acid, Aspirin, Benorylate, Biphenyl Aspirin, Bromosaligenin, Calcium Acetylsalicylate, Diflunisal, Etersalate, Fendosal, Flufenisal, Gentisic Acid, Glycol Salicylate, Imidazole Salicylate, Lysine Acetylsalicylate, Mesalamine, Morpholine Salicylate, 1-Naphthyl Sallicylate, Olsalazine, Parsalmide, Phenyl Acetylsalicylate, Phenyl Salicylate, 2-Phosphonoxybenzoic Acid, Salacetamide, Salicylamide O-Acetic Acid, Salicylic Acid, Salicyloyl Salicylic Acid, Salicylsulfuric Acid, Salsalate and Sulfasalazine), thiazinecarboxamides (e.g., Droxicarn, Isoxicam, Piroxicam and Tenoxican), ε-Acetamidocaproic Acid, S-Adenosylmethionine, 3-Amino-4-hydroxybutyric Acid, Amixetrine, Bendazac, Benzydamine, Bucolome, Difenpiramide, Ditazol, Emorfazone, Guaiazulene, Ketorolac, Meclofenamic Acid, Mefenamic Acid, Naburnetone, Nimesulide, Orgotein, Oxaceprol, Paranyline, Perisoxal, Pifoxime, Piroxicam, Proquazone, Tenidap and a COX-2 inhibitor (e.g., Rofecoxib, Valdecoxib and Celecoxib).

Further examples of anti-inflammatory agents include aspirin, a sodium salicylate, choline magnesium trisalicylate, salsalate, diflunisal, sulfasalazine, olsalazine, a para-aminophenol derivatives, an indole, an indene acetic acid, a heteroaryl acetic acid, an anthranilic acid, an enolic acid, an alkanones, a diaryl-substituted furanone, a diaryl-substituted pyrazoles, an indole acetic acids, or a sulfonanilide.

When treating demyelinating conditions, for example axonal degeneration, the compounds of the present invention can be administered in combination with immunotherapeutic agents such as interferons and anti-integrin blocking antibodies like natal izumab.

Examples of agents suitable for treating demyelinating disorders include Pirfenidone, Epalrestat, Nefazodone hydrochloride, Memantine hydrochloride, Mitoxantrone hydrochloride, Mitozantrone hydrochloride, Thalidomide, Roquinimex, Venlafaxine hydrochloride, Intaxel, Paclitaxel, recombinant human nerve growth factor; nerve growth factor, ibudilast, Cladribine, Beraprost sodium, Levacecarnine hydrochloride; Acetyl-L-carnitine hydrochloride; Levocarnitine acetyl hydrochloride, Droxidopa, interferon alfa, natural interferon alpha, human lymphoblastoid interferon, interferon beta-1b, interferon beta-Ser, Alemtuzumab, Mycophenolate mofetil, Zoledronic acid monohydrate, Adapalene, Eliprodil, Donepezil hydrochloride, Dexanabinol, Dexanabinone, Xaliproden hydrochloride, interferon alfa-n3, lipoic acid, thioctic acid, Teriflunomide, Atorvastatin, Pymadin, 4-Aminopyridine, Fampridine, Fidarestat, Priliximab, Pixantrone maleate, Dacliximab, Daclizumab, Glatiramer acetate, Rituximab, Fingolimod hydrochloride, interferon beta-1a, Natalizumab, Abatacept, Temsirolimus, Lenercept, Ruboxistaurin mesilate hydrate, Dextromethorphan/quinidine sulfate, Capsaicin, Dimethylfumarate or Dronabinol/cannabidiol.

Further examples of pharmaceutical agents that can be co-administered with the compounds of formula (I) include:

T-cell receptor (TCR) Vβ6 CDR2 peptide vaccine consisting of TCR Vβ6, amino acid sequence 39-58, Leu-Gly-Gin-Gly-Pro-Glu-Phe-Leu-Thr-Tyr-Phe-Gln-Asn-Glu-Ala-Gin-Leu-Glu-Lys-Ser (SEQ ID NO:1);

Myelin basic protein immunogen peptide, aminoacid sequence 75-95, Lys-Ser-His-Gly-Arg-Thr-Gin-Asp-Glu-Asn-Pro-Val-Val-His-Phe-Phe-Lys-Asn-Ile-Val-Thr (SEQ ID NO:2);

Tiplimotide, myelin basic protein immunogen vaccine peptide, aminoacid sequence 83-99, D-Ala-lys-pro-val-val-his-leu-phe-ala-asp-ile-val-thr-pro-arg-thr-pro, (SEQ ID NO:3);

Myelin basic protein immunogen peptide, aminoacid sequence 82-98, Asp-glu-asp-pro-val-val-his-phe-phe-lys-asp-ile-val-thr-pro-arg-thr, (SEQ ID NO:4);

Adrenocorticotropic hormone (ACTH), Ser-Tyr-Ser-met-glu-his-phe-arg-try-gly-lys-pro-val-gly-lys-lys-arg-arg-pro-val-lys-val-tyr-pro-asp-gly -ala-glu-asp-glu-leu-ala-glu-ala-phe-pro-leu-glut-phe, (SEQ ID NO:5).

In some embodiments, compounds of formula (A) can be administered in combination with antivascular agents, in particular agents inhibiting the growth factor receptors, Epidermal Growth Factor Receptor (EGFR), Vascular Epidermal Growth Factor Receptor (VEGFR), and Fibroblast Growth Factor Receptor (FGFR). Examples of such agents include, Iressa, Tarceva, Erbitux, Pelitinib, AEE-788, CP-547632, CP-547623, Tykerb (GW-2016), INCB-7839, ARRY-334543, BMS-599626, BIBW-2992, Falnidamol, AG1517, E-7080, KRN-951, GFKI-258, BAY-579352, CP-7055, CEP-5214, Sutent, Macugen, Nexavar, Neovastat, Vatalanib succinate, GW-78603413, Lucentis, Teavigo, AG-13958, AMG-706, Axitinib, ABT-869, Evizon, Aplidin, NM-3, PI-88, Coprexa, AZD-2171, XL-189, XL-880, XL-820, XL-647, ZK-CDK, VEGFTrap, OSI-930, Avastin, Revlimid, Endostar, Linomide, Xinlay, SU-668, BIBF-1120, BMS-5826624, BMS-540215.

In some embodiments, compounds of formula (I) can be administered in combination with agents that affect T-cell homing, extravastion and transmigration. Examples of such agents include, FTY-720PKI-166, PTK-787, SU-11248.

In some embodiments, compounds of formula (I) can be administered in combination with agents inhibiting VLA-4. Examples of such agents include, Tysabri, Bio-1211. HMR-1031, SB-683698, RBx-4638,RO-0272441, RBx-7796,SB-683699, DW-908e, AJM-300, and PS-460644.

Daily dose of administration of the compounds of the present invention can be repeated, in one embodiment, for one week. In other embodiments, daily dose can be repeated for one month to six months; for six months to one year; for one year to five years; and for five years to ten years. In other embodiments, the length of the treatment by repeated administration is determined by a physician.

The invention is illustrated by the following examples, which are not intended to be limiting in any way.

### Exemplification

### Example 1

### Synthesis of Compounds

Commercial reagents were purchased from Aldrich Chemical Company (Milwaukee, Wis.). All commercial solvents and reagents were used without further purification. Column chromatography was performed on 70-230 mesh silica gel. Melting points were determined on an Electrothermal capillary melting point apparatus and are uncorrected. sup. 1H NMR spectra were recorded on a Varian VXR spectrometer operating at 400 MHz, using TMS as an internal standard. Elemental compositions were verified by elemental analysis (Galbraith Laboratories) or by ion trap and time of flight mass spectrometry with spectral deconvolution of parent and derived ions. Analyses were within ±0.4% of theoretical values for C, H, and N.

With reference to the substituted imidazoacridinone in Scheme 1 and more specifically to Structure S1.1 therein, the following is a representative synthesis:

### 6-Chloro-2-[(4-Fluorophenyl)Amino]-3-nitrobenzoic Acid

A mixture of 2,6-dichloro-3-nitrobenzoic acid (18.88 g, 0.08 mol), 4-fluoroaniline (26.8 g, 0.18 mol) and EtOH (50 ml) was refluxed for 30 hours. The solvent was evaporated, benzene (100 ml) and 2N aqueous NaOH (150 ml) were added to the residue, and the mixture was vigorously stirred for 1 hour. Undissolved material was separated by filtration, the aqueous layer was isolated, and traces of benzene were removed by partial evaporation. The solution was then made acidic by addition of concentrated hydrochloric acid. The resulting yellow precipitate was collected by filtration and washed with water (100 ml). After drying, the crude material was crystallized from toluene to give 15.36 g (62%) of 7: mp 216-220° C.

By this method, beginning with the appropriate anilines, the following compounds were also prepared: 6-chloro-2-(4-methylphenyl)amino-3-nitro-benzoic acid, 6-chloro-2-(4-methoxyphenyl)amino-3-nitrobenzoic acid, 6-chloro-2-(4-benzyloxyphenyl)amino-3-nitrobenzoic acid, 6-chloro-2-(3-methylphenyl)amino-3-nitrobenzoic acid, 6-chloro-2-(3-methoxyphenyl)amino-3-nitrobenzoic acid, 6-chloro-2-(4-cyanophenyl)amino-3-nitrobenzoic acid, 6-chloro-2-(3-cyanophenyl)amino-3-nitrobenzoic acid, 6-chloro-2-[4-(methoxycarbonyloxy)phenyl]amino-3-nitrobenzoic acid, 6-chloro-2-[4-(methanesulfonyl)phenyl]amino-3-nitrobenzoic acid, 6-chloro-2-[4-(trifluoromethoxy)phenyl]amino-3-nitrobenzoic acid, 6-chloro-2-(4-methylphenyl)amino-3-nitro-benzoic acid, 6-chloro-2-(4-methoxyphenyl)amino-3-nitrobenzoic acid, 6-chloro-2-(4-benzyloxyphenyl)amino-3-nitrobenzoic acid, 6-chloro-2-(3-methylphenyl)amino-3-nitrobenzoic acid, 6-chloro-2-(3-methoxyphenyl)amino-3-nitrobenzoic acid, 6-chloro-2-(4-cyanophenyl)amino-3-nitrobenzoic acid, 6-chloro-2-(3-cyanophenyl)amino-3-nitrobenzoic acid, 6-chloro-2-[4-(methoxycarbonyloxy)phenyl]amino-3-nitrobenzoic acid, 6-chloro-2-[4-(methanesulfonyl)phenyl]amino-3-nitrobenzoic acid, 6-chloro-2-[4-(trifluoromethoxy)phenyl]amino-3-nitrobenzoic acid, 6-chloro-2-[4-(t-butoxycarbonyloxy)trifluoromethoxy)phenyl]amino-3-nitrobenzoic acid and others.

### 5-Chloro-8-Fluoro-2-nitro-1H-acridin-6-one, (S1.1)

A mixture of 6-chloro-2-[(4-fluorophenyl)amino]-3-nitrobenzoic acid (12.39 g, 0.04 mol), chloroform (100 ml), and POCl₃ (60 ml, 0.64 mol) was stirred at reflux for 8 h. Solvents were removed under reduced pressure. To the residue was added 200 ml of a mixture of 1,4-dioxane and water (8:1), and the mixture was acidified with concentrated hydrochloric acid and stirred at reflux for 2 h. Water was added (200 ml) and the precipitate was collected by filtration and crystallized from N,N-dimethylformamide-water to give 10.2 g (87%) of lb as orange needles: mp 287-291° C.

By this method, but starting with the appropriate 6-chloro-2-arylanino-3-nitrobenzoic acids, and separating isomers by recrystalization and/or column chromatography where needed, the following were prepared: 5-chloro-8-methyl-2-nitro-1H-acridin-6-one, 5-chloro-8-methoxy-2-nitro-1H-acridin-6-one, 5-chloro-8-benzyloxy-2-nitro-1H-acridin-6-one, 5-chloro-9-methyl-2-nitro-1H-acridin-6-one, 5-chloro-9-methoxy-2-nitro-1H-acridin-6-one, 5-chloro-8-cyano-2-nitro-1H-acridin-6-one, 5-chloro-9-cyano-2-nitro-1H-acridin-6-one, 5-chloro-8-methoxycarbonyloxy-2-nitro-1H-acridin-6-one, 5-chloro-8-methanesulfonyl-2-nitro-1H-acridin-6-one, 5-chloro-8-trifluoromethoxy-2-nitro-1H-acridin-6-one, 5-chloro-8-t-butoxycarbonyloxy-2-nitro-1H-acridin-6-one, and others.

Each of these synthons were then reacted with appropriate alkylamino-alkylamines to displace the 5-chloro-functionality in structures belonging to the S1.1 cohort, thus affording the corresponding cohort of S1.2 analogs. The nitro group in each of these was reduced to the S1.3 set of structures and these in turn were cyclo-annulated with formic acid, acetic acid and other acyl donors to afford the final imidazo acridinones, as per the structure indicated a S1.4 in Scheme 1 and also as formula (XXX).

Compounds prepared in this manner afford crystaline salts as the free base or as the hydrochloride salts. Isomeric impurities may first be removed by flash chromatography on silica gel with mixtures of methanol and dimethyl formamide as diluent. Purity is then established by HPLC with the following protocol:

GRADIENT CONDITIONS

Instrument Specifics:
Waters HPLC system
2795 separations module equipped with a
2474 Variable Fluorescence detector.
2996 PDA detector.
Column:
Supelco Discovery RP Amide C16, 3.0 × 125 mm, 5um or
Alltech Alltima C18, 3.2 × 150 mm, 5 μm
Column temperature: 30° C.
Sample temperature: 15° C.
Detector Conditions:
Fluorescence detector: excitation 420; emission 520 (Note:
Fluorescence detection is not used as this concentration saturates
the detector.)
PDA wavelength: 254
Gradient:
Mobile phase
A: 0.025M Ammonium formate, pH 4
B: Acetonitrile

Gradient

| Time | % A | % B | Curve |
|---|---|---|---|
| Initial | 90 | 10 | — |
| 30 m | 30 | 70 | 6 (linear) |
| 31 m | 90 | 10 | 6 |

Flow rate: 0.7 ml/min
Run time: 30 min with 5 min equilibration between injections

All of the compounds prepared by the sequence of steps exemplified here proved homogenous by HPLC, with retention times of 7-9 minutes. The precursors from Steps S1.1 through S1.3 and various impurities show retention times less than 6 minutes or greater than 12 minutes. The final purity of these materials was established as greater than 98%, in order to qualify for biological testing. NMR, elemental compositions and mass spectral properties conformed to theory.

### Example 2

### Determination of FLT3 and Related Protein Tyrosine Kinase Activity Targeting In Vitro

Protein tyrosine kinases are major biological effectors and because of their central role in regulatory signal transduction they have become target for drug development. The structures of all major kinases and their mutants are known, have been cloned and the functional domains, e.g. the juxtamembrane, ATP and catalytic components projecting from the cell membrane into the cytosol have been expressed through recombinant genetic techniques. The recombinant enzymes have been assembled into test panels to exploit the fact that in the presence of ATP they will phosphorylate an appropriate substrate and the phosphorylation rate and extent can then be measured via an optical reporter system. This testing stratagem and its application in drug development has been discussed in a review by M Vieth et al. (Drug Disc. Today 10: 839-846, 2005). The translation of in vitro results and the correlation to in vivo cellular assays is considered to be high and has been validated in cellular models, for example, in the work of J. S. Melnik et al. (Proc. Nat. Acad. Sci. 103:3153-3158, 2006).

Testing of the compounds described in this invention was carried out using the SelectScreen™ platform from Invitrogen, Inc. (Carlsbad, Calif., USA) and the details of its performance are readily viewed via the web by linking to: http://www.invitrogen.com/downloads/SelectScm_Brochure.pdf.

Briefly the approach is based on treating each specific kinase with a unique substrate and optical reporter system in the presence of ATP at 100 micromolar or at the apparent optimal ATP concentration for each kinase. In controls, the substrate is phosphorylated and a baseline optimal response is recorded. Graded amounts of putative inhibitor are then added in separate increments to generate a dose response curve. The latter is obtained by fitting to a sigmoid saturation equation, such as the Hill equation, and the concentration of test article producing 50 per cent inhibition is then noted as the IC₅₀. An effective level of inhibition in the low nanomolar range is considered to qualify the test compound as potential drug or targeting agent against the specific kinase that it has inhibited. The IC₅₀ value is, therefore, a measure of potency. Another important feature is specificity. It is considered a desirable property when claiming efficacy to determine how many kinases are inhibited by the same molecule. The fewer number inhibited points toward specificity; the greater to inhibitory promiscuity.

For the compounds of this invention, the in vitro kinase screens revealed not only high activity against FLT3 and its constitutively activate mutant FLT3 D835Y but also a high, and unprecedented specificity, with at least a 2-log unit difference between the EC50 value for FLT3 and the corresponding inhibitory index for other and closely 25 related protein tyrosine kinases, e.g. KDR, cKIT, PDGF, FGF, NTRK and others, in the TK and RTK domains.

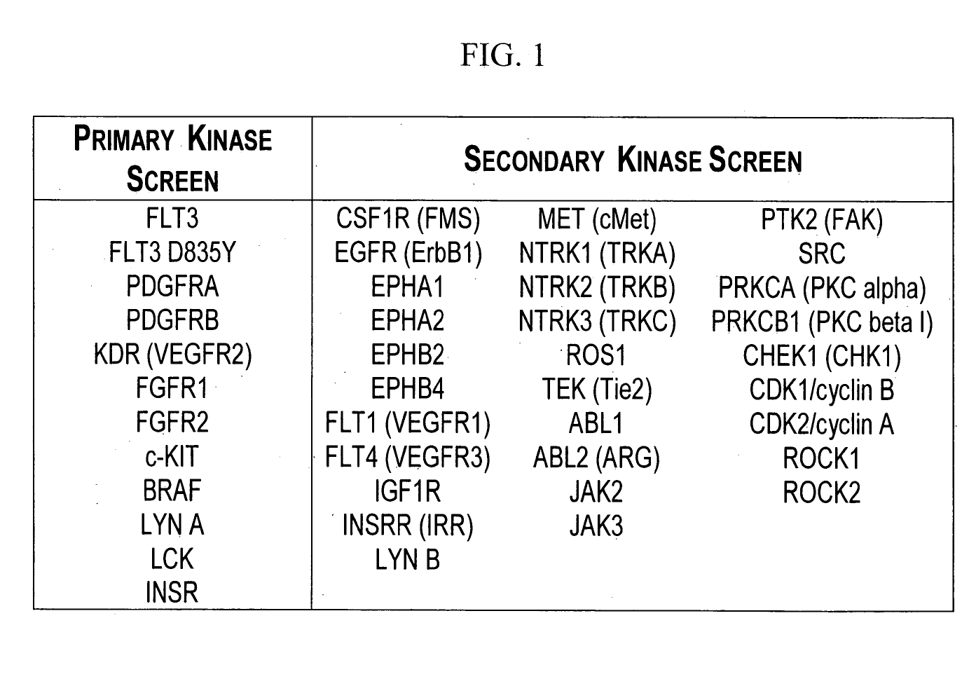

The screening was carried out in two phases as shown in FIG. 1. First the compounds of this invention were tested against a subset, the primary kinase screen, to determine initial activity at 1 and 0.1 micromolar concentrations. If greater than 50% inhibition was observed (at 100 mM ATP) against all but INSR, the compounds proceeded to be tested against the broader Secondary Kinase Screen. Thereafter compounds with at least 50% inhibitory activity on any given kinase were re-examined with a 9 point dilution series to obtain the EC₅₀ against that kinase. Note should be made that activity against INSR, the insulin receptor, in the primary screen or against INSRR (insulin receptor-related receptor) or IGF1, the insulin like growth factor 1, in the secondary screen would have disqualified the compounds from further development, because inhibition of an essential homeostatic set of receptors is a hallmark of undesirable side effects. None of the compounds of this invention inhibited these so called “housekeeping” receptors even at 10 micromolar concentration, a high enough concentration generally indicative of drug safety absent.

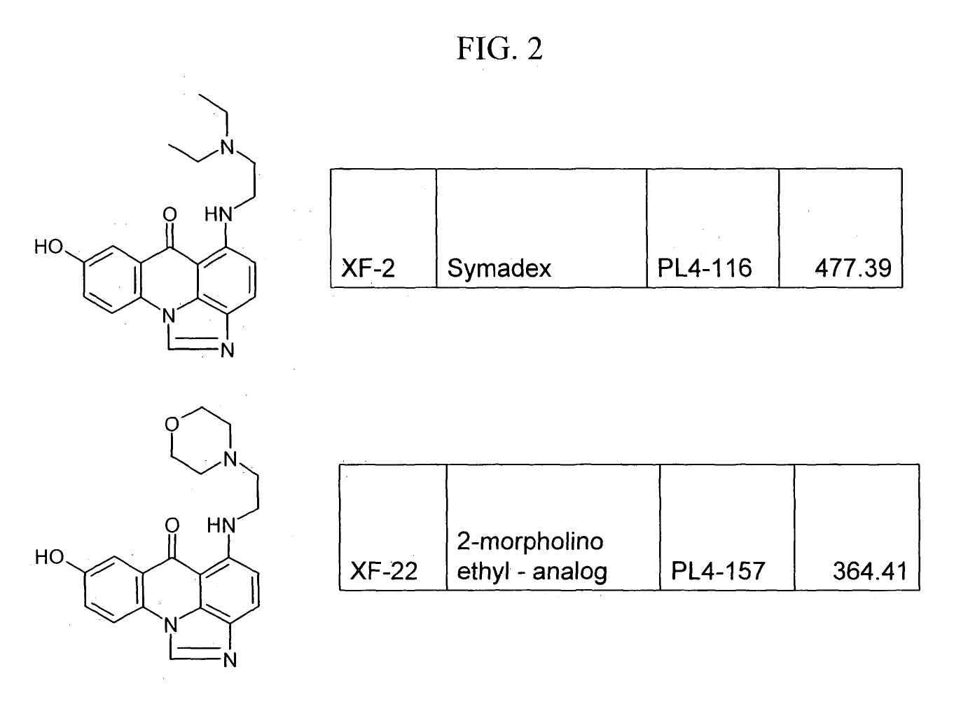

With reference to formula (XXX), two such examples of novel FLT3 inhibitors conforming to Formula (I), shown in FIG. 2:
a compound represented by the following structural formula (also referred to as XF-2):

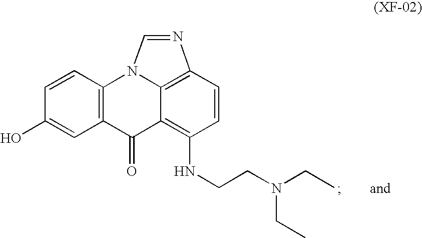

a compound represented by structural following structural formula (also referred to as XF-22):

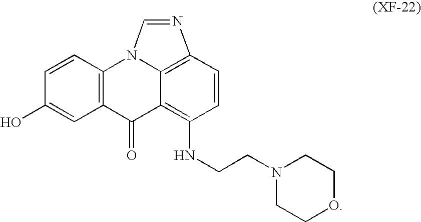

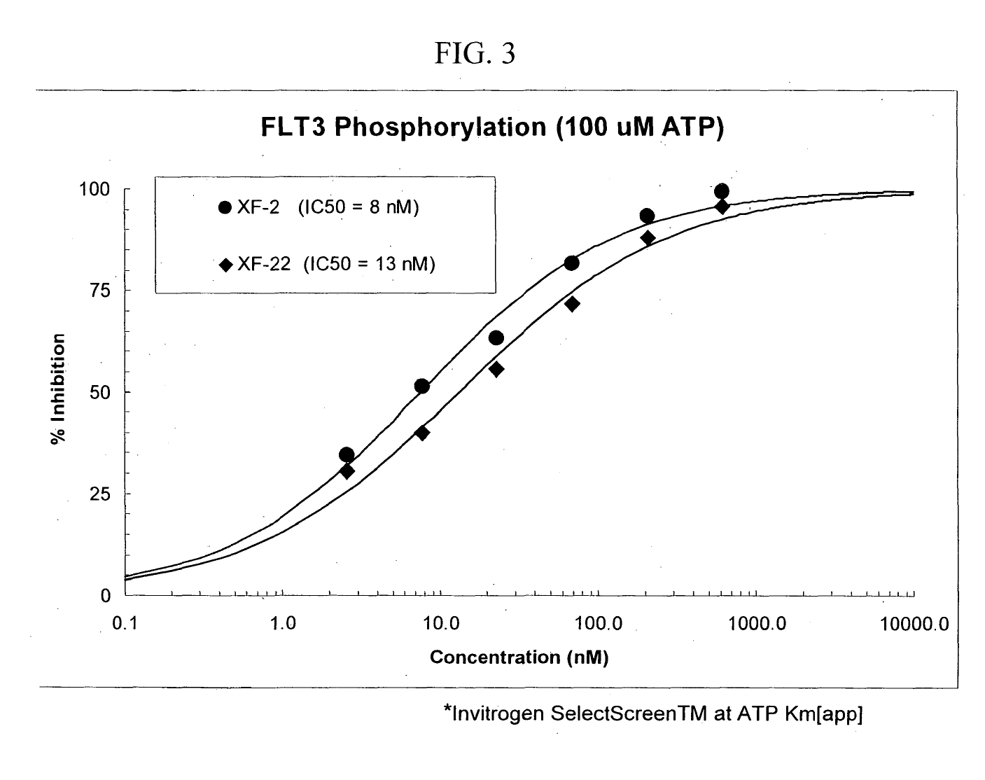

FIG. 3 shows their activity as FLT3 inhibitors; a high degree of inhibition is noted, with IC₅₀ values of 8 and 12 nM, respectively.

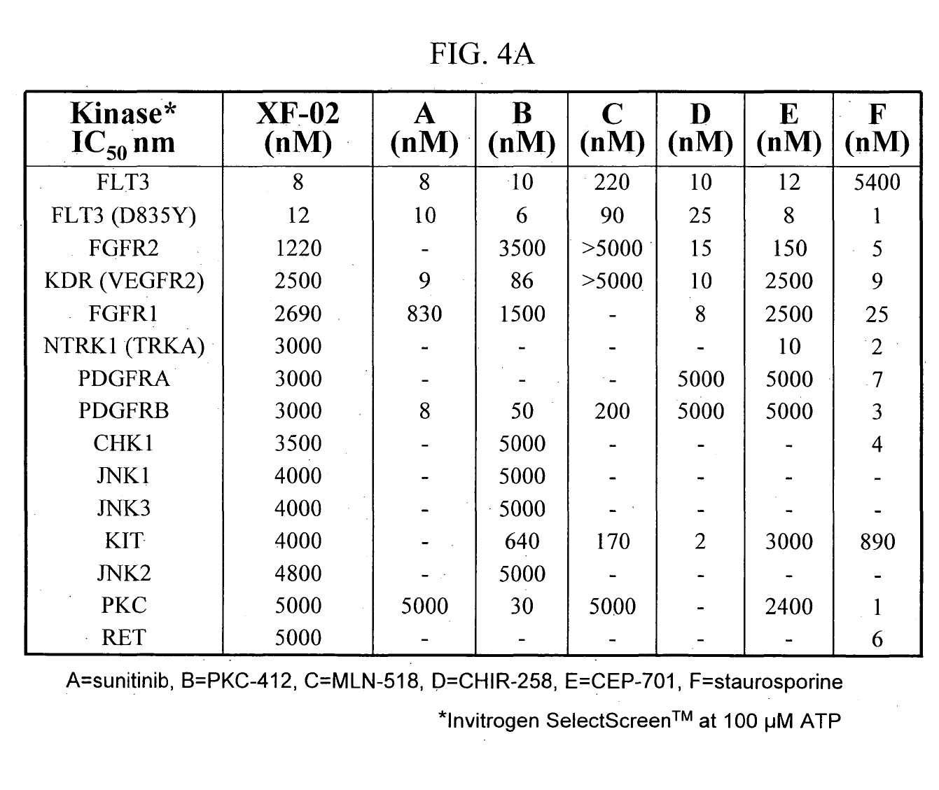

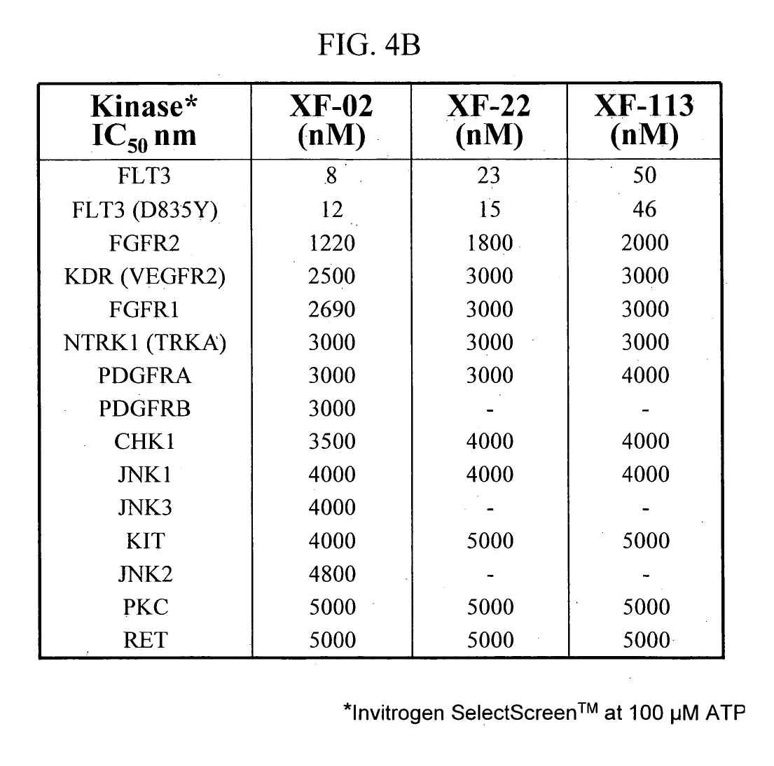

As shown in the table presented in FIG. 4A and FIG. 4B, a detailed analysis of the specificity of compound XF-02, XF-22, and XF-113

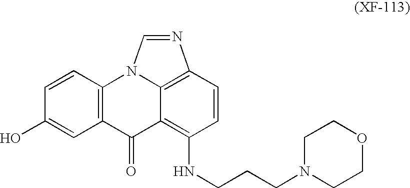

contrasts its performance as a selective FLT3 inhibitor against the activity of a representative selection of kinase inhibitors in current development or recently approved as therapeutic drugs (see FIG. 4A). Two key novel features emerge from these data:
1) XF-2, XF-22, and XF-113 are active against FLT3 and its mutant form and the activity profile drops off precipitously as one explores additional RTK and TK enzymes. In the case of FGFR2, the fibroblast growth factor receptor 2, a close neighbor in the “kinome tree” to FLT3, the inhibitory activity of XF-2, for example, drops 100 fold by two orders of magnitude in EC₅₀ value.
2) The inhibitory activity of XF-2, XF-22, and XF-113 is many fold less pronounced against other closely related targets, in particular the KDR, PDGF and TRK members of the RTK family, which are co-inhibited by other more promiscuous inhibitors that have been promulgated as FLT3 inhibitor.

It is evident that none of the compounds exemplified in FIGS. 4A other than the compound of the present invention (XF-02) are truly selective against FLT4. The two such references with the closest profile to XF-2 are PKC-412 and CEP-701. The former cross-reacts with KDR, PDGFRb, and PKC. The latter, inhibits FGFR2 and, more significantly, the neurokinin receptor NTRK1.

The contrast in specificity profile between XF-2 and CEP-701 takes on added significance, because CEP-701 has been implicated as a therapeutic agent in dendritic cell biology and subsequent treatment of inflammatory, immunocompromising, degenerative and demyelinating disorders, which is the subject of this invention as described by K. A. Whartenby et al. (Proc. Nat. Acad. Sci. 102: 16741-16746, 2005) and D. Small et al. (Patent Application WO 2006/020145 A2, 23 Feb. 2006).

It should be noted in this context that the activity profile of XF-22 also matched that of XF-2 in contrast to CEP-701 and PKC-412, indicating that high selectivity is a novel and unexpected feature of the imidazoacridinone FLT3 inhibitors.

### Example 3

### Determination of FLT3 and Related Protein Tyrosine Kinase Activity Targeting in Cell Culture

It is now understood that an important subset of human myeloid leukemia cells overexpress FLT3 as constitutively activated mutations. The most prevalent of these is the internal tandem duplication. Two cell lines commonly used to test the potency of FLT3 inhibitors on cell growth and viability are the RS4(11) and MV4(11). The RS4(11) cell line, also known as RS4;11, was established from a bone marrow patient with acute lymphoblastic leukemia. This female patient was 32 years of age. The cells lack surface and cytoplasmic immunoglobulin, and are negative for CD10. The cells have a characteristic chromosome translocation (4;11)(q21;q23), and an isochromosome for the long arm of chromosome 7. These cells express the wild-type FLT3 receptor and are further characterized by expression of the MHC Class II antigens (HLA DR+); CD9+; CD24+. The MV4(11) cell line, also known as MV-4-11, was established from the blast cells of a 10 year old male with biphenotypic B myelomonocytic leukemia (ATCC). Other sources indicate this cell line is derived from acute monocytic leukemia (AML FAB M5). The cytogenetic analysis reveals that there are 48 chromosomes (+8, +19) and a (4;11)(q21;q23) translocation. These cells express a mutant form of FLT3 containing an internal tandem duplication (ITD), and are further characterized by expression of CD4 (40-96%); CD10 (4-11%); CD15 (96-99%).

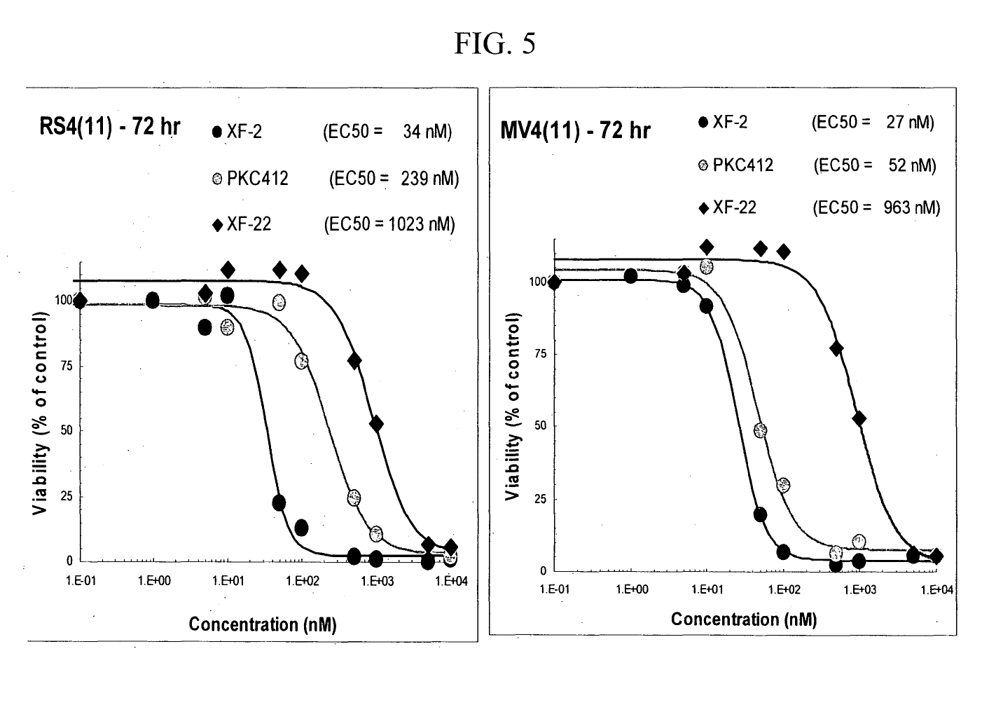

When standard protocols for adherent cell culture are followed, according to the instructions supplied by the American Type Culture Collection for the two line, the cell will proliferate with an approximate 30 hour doubling time. However, when exposed to graded amounts of XF-2 or SF-22, the growth and viability of these cell lines is arrested. As shown in FIG. 5, the effective concentration to decrement cell viability by 50% (EC₅₀) for XF-2 in RS4(11) at 72 hrs of continuous exposure is 34 nM, which compares favorably to the positive control, PKC-412, a known FLT3 inhibitor. The EC₅₀ for XF-22 fell closer to the micromolar mark, or a third less potent than PKC-412. A similar result was obtained in the more clinically significant MV4(11) line, which bears the internal tandem duplication and requires an active FLT3 signaling cascade for survival. In this case XF-2 and PKC412 were closely matched, while XF-22 showed less activity. When the drugs were used to arrest growth in a the HL-60 leukemia, which is known to neither express FLT3 nor require the FLT3 signaling cascade for growth or viability, the EC₅₀ of both XF-2 and XF-22 rose to values greater than 10 micromolar, as did PKC412, confirming that their cytotoxicity had been derived in large measure by attack on FLT3.

### Example 4

### Treating with FLT3 Inhibitors Abrogates the Disease Course of Experimental Autoimmune Encephalomyelitis (EAE)

EAE has become a benchmark model for the development of effective treatments applicable to both the remitting-relapsing and the secondary progressive forms of multiple sclerosis is humans. It is now believed that the EAE process is mediated by autoreactive cells through a signaling cascade that “instructs” them to attack myelin. The training of autoreactive cells to assault myelin is mediated by antigen presenting cells, principally dendritic cells, and microglia in the nervous system. This training process is influenced by the presence of FLT3 ligand, as a growth factor, and by a functionally active FLT3 receptor and signaling pathway that controls maturation of the antigen presenting cells and also the maturation of macrophages responsive to them. This interaction system has been described recently in detail by B. Pulendran (Nature Immunol 7: 699-700, 2006) and by N. Onai et al. (J. Experimental Med. 203:227-238, 2006.

It is acknowledged in similar reviewers of the literature that the interaction of FLT3, dendritic cells and macrophages plays a key role in the pathogenesis of other autoimmune diseases, including arthritis, inflammatory bowel disease and atherosclerotic plaque (C. Reis e Sousa, Nature Reviews Immunology 6:476-483, 2006). Given the recent nature of these findings, the medicinal chemistry of compounds affecting FLT3 as potential therapies for autoimmune disease is in its relative infancy. The immediate connection between FLT3 inhibition and abrogation of the disease course was recently demonstrated by the aforementioned investigators, K. A. Whartenby et al. (Proc. Nat. Acad. Sci. 102: 16741-16746, 2005) and D. Small et al. (Patent Application WO 2006/020145 A2, 23 Feb. 2006). They used the drug CEP-701, a structural relative of PKC-412, which has been used in these examples as a benchmark for FLT3 inhibition.

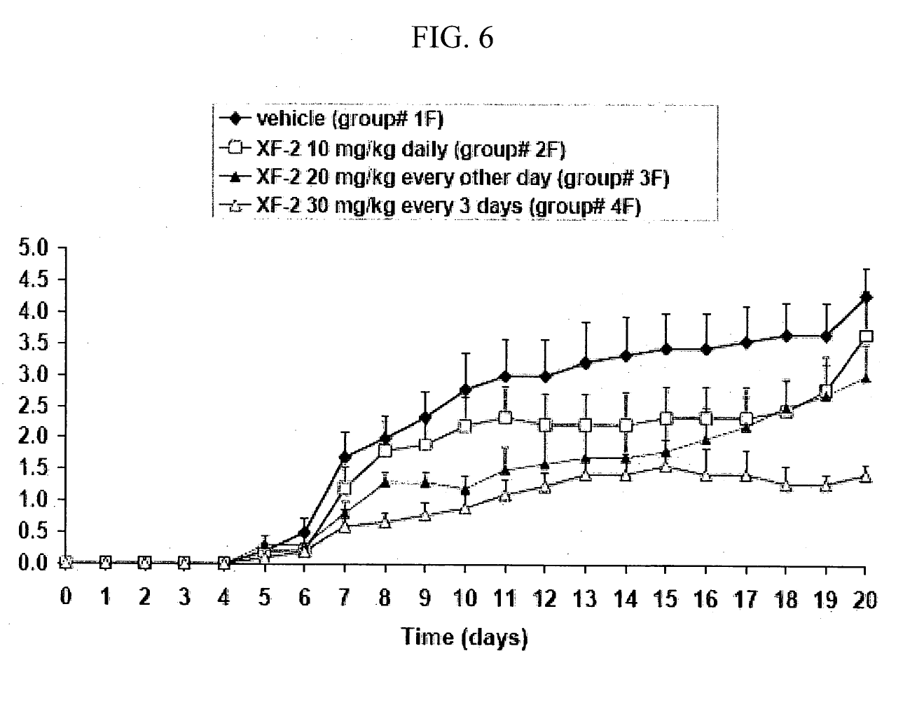

The EAE preparation used to test the efficacy of the imidazoacridinone analogs of the present invention followed a similar protocol as described by Small et al. (Patent Application WO 2006/020145 A2, 23 Feb. 2006) and was conducted by MD Biosciences, a contract research organization that provides in vivo and in vitro disease models as well as mode of action studies for general inflammatory screening, arthritis, IBD, and multiple sclerosis. C57BL/6 mice were immunized with proteolipid protein emulsified in Complete Freund's Adjuvant on day 0 of the study. After a booster with pertussis toxin also on day 0 and then on day 2, the mice were treated on day 5 after disease induction with three oral dosing schedules of XF-2, at 10 mg/kg every 24 hrs, 20 mg/kg every 48 hrs and 30 mg/kg every 72 hrs. FIG. 6 shows graphically that the mice treated with XF-2 (FLT3) inhibitor have lower clinical scores indicative of disease amelioration, and these differ significantly from the scores of matched vehicle controls exhibiting the full course of disease.

Taken as a whole, this example, and the preceding ones on the efficacy of FLT3 inhibition indicate that the imidazoacridinone of this invention not only inhibit FLT3 in vitro and in isolated cells, but also in a relevant disease model.

### Example 5

### Synthesis of the Compounds of the Invention

Compounds of formula (I) can be synthesized according to the following synthetic scheme.

### 1) 2-Fluoro-5-nitro-6-(1H-indazol-5-ylarnino)benzoic acid

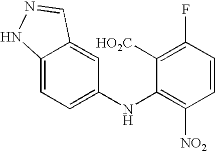

3-Nitro-2,6-difluorobenzoic acid (20.0 g, 98.44 mmol) was added to a solution of ethanol (100 mL) and water (100 mL). The acid solution was cooled to 10° C. and triethylamine (25.09 mL) added dropwise under rapid stirring to ensure the temperature did not exceed 40° C. 5-Aminoindazole (13.01 g, 98.44 mmol) was then added in portions and the combined mixture heated to 70° C. for 16 hours. A solution of water (100 mL) and conc. HCl (100 mL) was made, heated to 60° C. and placed under vigorous stirring. The reaction mixture, still at 70° C., was transferred to the HCl/water solution in small portions and allowed to cool to room temperature. The mixture was stirred for a further 4 hours to ensure maximum precipitation. The resulting precipitate was filtered off and washed with water (2×60 mL) and dried in a vacuum oven overnight (28.3 g, 89.56 mmol, 94%). ¹H δ (d6-DMSO): 6.97 (1H, dd, ArH, J=8.8, 2.4 Hz), 7.18 (1H, d, ArH, J=2.4 Hz), 7.43 (1H, d, ArH, J=8.8 Hz), 7.45 (1H, d, ArH, J=8.8 Hz), 8.10 (1H, d, ArH, J=8.8 Hz), 8.99 (1H, s, ArH), 9.15 (1H, s, NH), 9.87 (1H, s, NH); HPLC: R₁=3.52 min.; LRMS: m/z=315.4 (M−H)

### 2) 1-Fluoro-4-nitropyrazolo[7,8a]-acridin-9(10H)-one

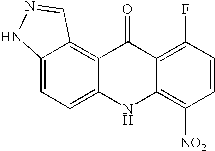

A suspension of 2-fluoro-5-nitro-6-(1H-indazol-5-ylamino)benzoic acid (20.0 g, 65.096 mmol) in chloroform under argon had freshly distilled POCl₃ (24.3 mL, 260.383 mmol) added to it. The mixture was heated to 80° C. for 16 hours. Once cooled, ethanol (50 mL) was added slowly to quench the excess POCl₃ reaction and then stirred for 30 minutes at room temperature. At this point, the mixture was reduced to dryness under vacuum. Water (100 mL) was added to the residue and saturated sodium bicarbonate solution added until the pH=8. The mixture was then stirred for 60 minutes at room temperature and the precipitate filtered off, washed with water (2×80 mL) and dried overnight in a vacuum oven (19.219 g, 64.445 mmol, 99%). ¹H δ (d6-DMSO): NMR unavailable since compound very insoluble; HPLC: R₁=6.22 min.; LRMS: m/z=299.1 (M+H)

### 3) Compound (A): 1-Fluoro-4-nitropyrazolo[7,8a]-imidazo[4,5,1-de]-acridin-9(10H)-one

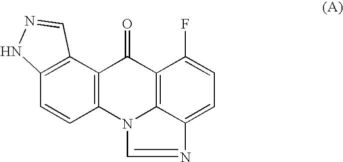

To a slurry of starting material (7.112 g, 23.846 mmol) in formic acid (75 mL), was added SnCl₂.2H₂O (23.07 g, 102.25 mmol) in conc. HCl (15 mL). The combined mixture was stirred at room temperature for 30 minutes then heated to 95° C. for 20 hours. Once cooled, the solid was filtered off and partially dried before being slurried in sat. sodium bicarbonate solution and stirred for 60 mins. The precipitate was then filtered off and washed with water (2×100 mL) and dried in a vacuum oven overnight (6.303 g, 22.654 mmol, 95%).

### 4) Compound (B)

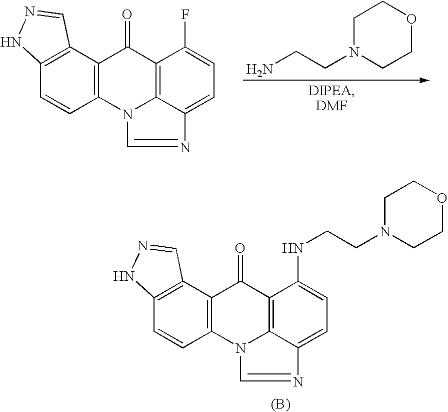

Amide core (0.294 g, 1.057 mmol), 2-morpholine ethylamine (0.220 g, 1.961 mmol, 0.222 mL), diisopropylethylamine (0.273 g, 2.114 mmol, 0.368 mL) and dimethylacetamide (2.0 mL) were combined in a 5 mL reaction tube and heated to 150° C. under microwave irradiation for 20 minutes. Once cooled, the solvent was removed and the residue columned over silica gel (0-20% MeOH in CHCl₃). The desired fractions were pooled and the solvent removed to yield the target compound. ¹H δ (d6-DMSO):

2.41 (2H, m, CH₂NCH₂), 2.68 (2H, m, CH₂), 3.19 (2H, m, CH₂NCH₂), 3.36 (2H, m, NHCH₂), 3.63 (4H, m, CH₂O CH₂), 6.89 (1H, d, ArH, J=8.6 Hz), 8.01 (1H, d, ArH, J=8.6 Hz), 8.16 (1H, d, ArH, J=9.0), 8.44 (1H, d, ArH, J=9.0 Hz), 9.00 (1H, s, Pyrazole-H), 9.07 (1H, m, NH), 9.29 (1H, s, Imidazole-H), 13.69 (1H, s, N═NH); HPLC: R₁=3.55 min.; LRMS: 389.0 (M+1).

Additional compounds with the fused pyrazole scaffold, for example, compounds (C) and (D),

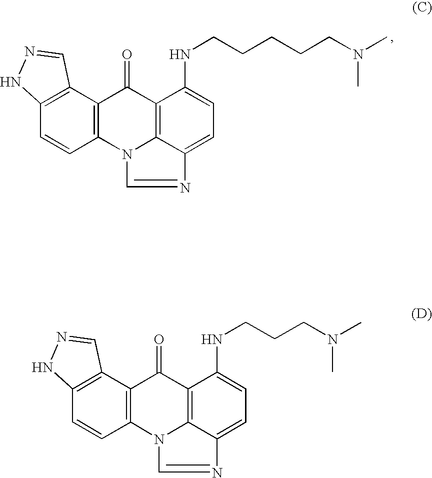

are prepared in a similar manner, following the method used for (B), namely, by treating the amide core, compound (A), with other dialkylamino-alkyl amines and the same reaction stoichiometry used in the model reaction with 2-morpholine ethylamine. For example, compounds (C) and (D) were prepared by the addition of 5-N,N-dimethylamino pentylamine and 3-N,N-dimethylamino propylamine to compound (A). The resulting compounds conformed to theory as evidenced by their NMR and mass spectroscopic features, shown in the accompanying table that provides a handy comparison of the analytical properties for the precursor amide core, compound (A), and it derivatives.

### Spectroscopic Data on the Pyrazole Core and Additional Analogs Prepared in the Same Manner as XF-122

| | | | | LRMS | HPLC |
|---|---|---|---|---|---|
| Structure | XF- | Lot No. | ¹H NMR (δ) | (m/z) | (Rₜ) |
| 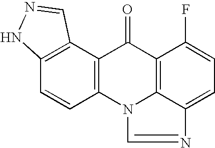 | 120 | KD02- 008 | 7.45 (1H, dd, ArH, J = 8.8, 8.8 Hz), 8.20 (1H, d, ArH, J = 9.2 Hz), 8.24 (1H, dd, ArH, J = 8.8, 3.4), 8.44 (1H, d, ArH, J = 9.2 Hz), 8.96 (1H, s, ArH), 9.64 (1H, s, ArH), 13.77 (1H, s, NH) | 279.0 (M + H) | 5.27 min. |
| 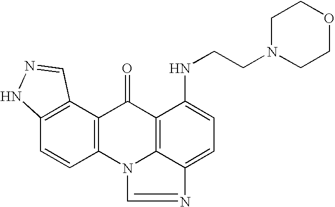 | 122 | KD01- 148 | 2.4 (2H, m, CH₂NCH₂), 2.68 (2H, m, CH₂), 3.19 (2H, m, CH₂NCH₂), 3.36 (2H, m, NHCH₂), 3.63 (4H, m, CH₂OCH₂), 6.89 (1H, d, ArH, J = 8.6 Hz), 8.01 (1H, d, ArH, J = 8.6 Hz), 8.16 (1H, d, ArH, J = 9.0 Hz), 8.44 (1H, d, ArH, J = 9.0 Hz), 9.00 (1H, s, ArH), 9.07 (1H, m, NH), 9.29 (1H, s, ArH), 13.69 (1H, s, N═NH) | 389.0 (M + H) | 3.52 min. |
| 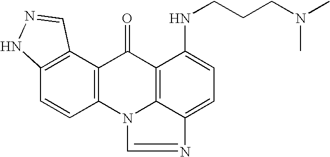 | 123 | KD01- 149 | 2.09 (2H, t, CH₂, J = 7.2 Hz), 2.75 (6H, s, N(CH₃)₂, 3.18 (2H, t, NCH₂, J = 7.2 Hz), 3.54 (2H, m, NHCH₂), 6.90 (1H, d, ArH, J = 9.2 Hz), 8.03 (1H, d, ArH, J = 9.2 Hz), 8.17 (1H, d, ArH, J = 9.2 Hz), 8.44 (1H, d, ArH, J = 9.2 Hz), 8.99 (1H, s, ArH), 9.03 (1H, m, NH), 9.30 (1H, s, ArH), 12.76 (1H, s, NH) | 361.0 (M + H) | 3.56 min. |
| 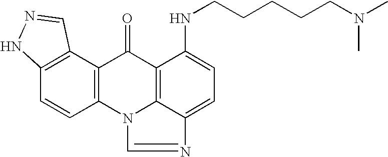 | 124 | KD01- 150 | 1.32 (2H, m, CH₂), 1.56 (2H, m, CH₂), 1.64 (2H, m, CH₂), 2.66 (6H, s, N(CH₃)₂), 2.74 (2H, m, CH₂), 2.93 (2H, m, CH₂), 6.86 (1H, d, ArH, J = 8.8 Hz), 8.01 (1H, d, ArH, J = 8.8 Hz), 8.16 (1H, d, ArH, J = 8.8 Hz), 8.45 (1H, d, ArH, J = 8.8 Hz), 8.99 (1H, s, ArH), 9.02 (1H, m, NH), 9.30 (1H, s, ArH) | 389.0 (M + H) | 4.67 min. |

### Example 6

### Synthesis of the Compounds of the Invention

Representative compounds of formula (I) can be synthesized according to the following synthetic schemes. Compound XF-02, represented by a formula shown in Example 2, is the starting compound in each of the schemes below. XF-02 can be synthesized according to a method disclosed in U.S. Pat. No. 6,229,015. The aforementioned patent is incorporated herein by reference.

### Compound (E): Acetic acid 5-(2-diethylamino-ethylamino)-6-oxo-6H-2,10b-diaza-aceanthrylen-8-yl ester

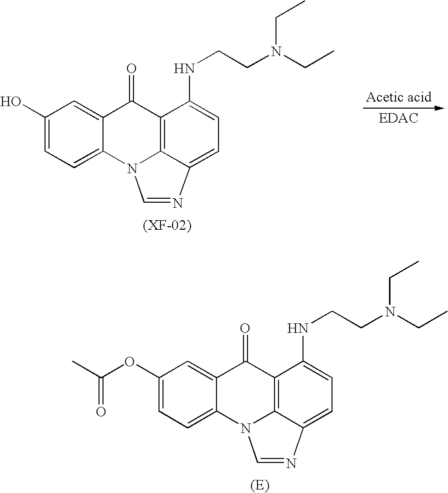

A solution of (XF-02) free base (2.11 g, 6.02 mmol), N-(3-dimethylaminopropyl)-N′-ethylcarbodiimide hydrochloride (1.73 g, 9.03 mmol), 4-(dimethylamino)pyridine (12 mg, 0.10 mmol), acetic acid (0.523 mL, 9.03 mmol), and N,N-diisopropylethylamine (2.35 mL, 13.5 mmol) in anhydrous DMF (36 mL) was stirred at room temperature for 20 hours. The mixture was concentrated to give a powder, which was subject to chromatograsphy (5-10% methanol in chloroform, silica gel) to give the title compound (1.83 g, 77% yield) as a bright yellow solid.

¹H NMR δ (CD₃SOCD₃): 9.13 (1H, s), 8.93 (1H, t, J=4.7 Hz), 8.42 (1H, d, J=8.6 Hz), 8.04 (1H, d, J=2.7 Hz), 7.94 (1H, d, J=9.0 Hz), 7.68 (1H, dd, J=9.0, 2.7 Hz), 6.76 (1H, d, J=9.0 Hz), 3.39-3.33 (4H, m), 2.55 (4H, q, J=7.0 Hz), 2.32 (3H, s), 1.01 (6H, t, J=7.0 Hz). ¹³C NMR δ (CD3SOCD₃): 177.40, 170.05, 149.97, 148.35, 136.09, 133.14, 133.08, 131.06, 129.95, 128.29, 126.51, 120.51, 118.35, 107.50, 102.75, 51.80, 47.30, 41.04, 21.70, 12.40. LRMS: m/z =393.0 (M+H)

### Compound (F). Octanoic acid 5-(2-diethylamino-ethylamino)-6-oxo-6H-2,10b-diaza-aceanthrylen-8-yl ester

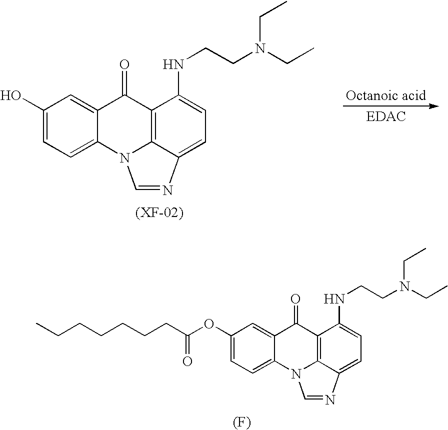

To a stirred solution of (XF-02) free base (3.50 g, 10 mmol), octanoic acid (1.59 mL, 10 mmol), and N-(3-dimethylaminopropyl)-N′-ethylcarbodiimide hydrochloride (2.30 g, 12 mmol) in DMF (60 mL) were added 4-(dimethylamino)pyridine (12 mg, 0.10 mmol) and triethylamine (2.83 mL, 20 mmol). The reaction mixture was stirred at ambient temperature for 66 hours (over weekend) and then concentrated to yield a solid, which was purified by chromatography 6-20% methanol in chloroform, silica gel) furnished the title compound (4.25 g, 89% yield) as greenish yellow solid.

¹H NMR δ (CD₃SOCD₃): 9.14 (1H,s), 9.07 (1H, s), 8.43 (1H, d, J=8.6 Hz), 8.23 (1H, d, J=9.0 Hz), 8.02 (1H, d, J=2.3 Hz), 7.69 (1H, dd, J=9.0, 2.3 Hz), 6.67 (1H, d, J=9.0 Hz), 3.63-3.54 (2H, m), 3.40-3.36 (2H, m), 3.17-3.11 (2H, m), 2.70 (2H, t, J=6.2 Hz), 2.4-1.4 (12H, m), 1.00 (6H, t, J=7.8 Hz), 0.83 (3H, t, J=7.1 Hz).

LRMS: m/z=477.0 (M+H)

### Compound (G). 2,2-Dimethyl-propionic acid 5-(2-diethylamino-ethylamino)-6-oxo-6H-2,10b-diaza-aceanthrylen-8-yl ester

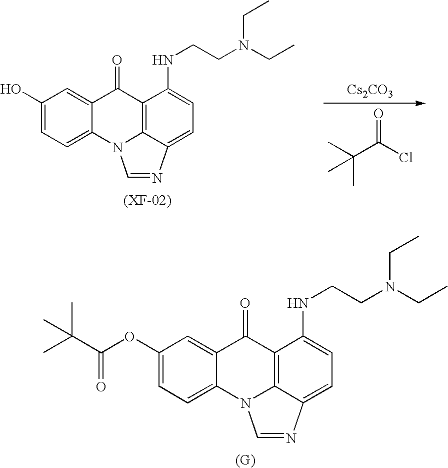

To a stirred solution of (XF-02) free base (0.280 g, 0.800 mmol) in anhydrous DMF (8 mL) was added cesium carbonate (0.67 g, 2.40 mmol). After 10 minutes, the initial yellow solution became orange. Trimethylacetyl chloride (0.295 mL, 2.40 mmol) was added via a syringe, the orange solution immediately turned greenish and cloudy. After 21 hours, the mixture was concentrated, the resulting solid was subject to chromatography (5-20% methanol in chloroform, silica gel) to give the title compound (0.13 g, 37% yield) as a yellow silid.

¹HNMR δ (CD₃SOCD₃): 9.15 (1H, s), 8.94 (1H, t, J=4.7 Hz), 8.45 (1H, d, J=9.0 Hz), 7.98 (1H, d, J=2.7 Hz), 7.95 (1H, d, J=8.6 Hz), 7.68 (1H, dd, J=9.0, 2.7 Hz), 6.77 (1H, d, J=9.0 Hz), 3.39 (2H, m), 3.30 (2H, m), 2.55 (4H, m), 1.35 (9H, s), 1.02 (6H, t, J=7.4 Hz). ¹³C NMR δ (CD₃SOCD₃): 177.36, 177.15, 149.96, 148.56, 136.09, 133.15, 133.06, 131.07, 129.95, 128.21, 126.52, 120.26, 118.40, 107.60, 102.75, 51.25, 46.97, 41.04, 39.33, 27.45, 12.51. LRMS: m/z=435.3 (M+H)

### Compound (J). Hexanoic acid 5-(2-diethylamino-ethylamino)-6-oxo-6H-2,10b-diaza-aceanthrylen-8-yl ester

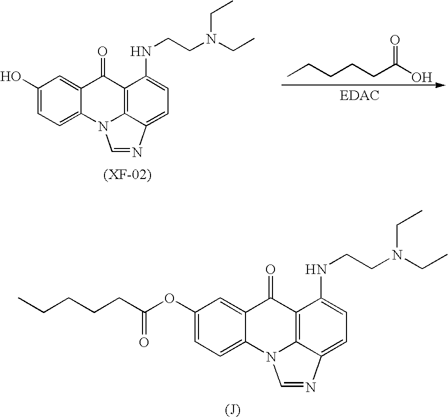

A solution of (XF-02) free base (2.20 g, 6.28 mmol), N-(3-dimethylaminopropyl)-N′-ethylcarbodiimide hydrochloride (1.33 g, 6.91 mmol), 4-(dimethylarnino)pyridine (12 mg, 0.10 mmol), hexanoic acid (0.866 mL, 6.91 mmol), and triethylamine (1.94 mL, 13.8 mmol) in anhydrous DMF (50 mL) was stirred at room temperature for 19 hours. The mixture was concentrated to give a powder, which was subject to chromatograsphy (10-20% methanol in chloroform, silica gel) to give the title compound (0.936 g, 30% yield) as a yellow solid.

### Compound (K). Butyric acid 5-(2-diethylamino-ethylamino)-6-oxo-6H-2,10b-diaza-aceanthrylen-8-yl ester

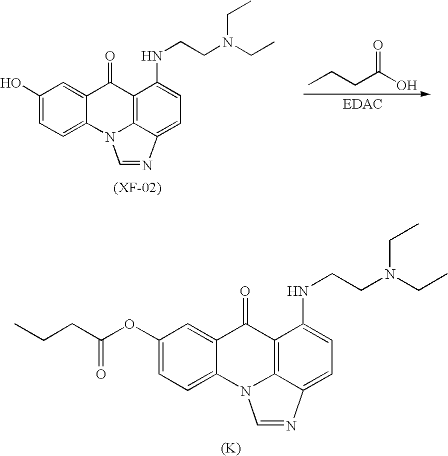

To a stirred solution of (XF-02) free base (3.01 g, 8.60 mmol) and butyric acid (0.870 mL, 9.46 mmol) in anhydrous DMF (36 mL) were added N-(3-dimethylaminopropyl)-N′-ethylcarbodiimide hydrochloride (1.81 g, 9.46 mmol), 4-(dimethylamino)pyridine (12 mg, 0.10 mmol), and triethylamine (4.21 mL, 30 mmol). The reaction mixture was stirred at RT for 65 hours (over weekend), and concentrated on a rotary evaporator. The resulting residue was purified by chromatography (10-20% methanol in chloroform, silica gel) to afford the title compound (2.25 g, 62% yield) as a yellow solid.

### Example 7

### Determination of FLT3 Tyrosine Kinase Activity Targeting In Vitro

The terms “IC50” and “EC50” are used interchangeably. As used herein, “EC50” refers to nMolar concentration at median percent inhibition determined by dose respose (DR) assay. As used herein, the term “E1000” refers to percent inhibition at 1000 nMolar determined by assay. EC50 was calculated based on dose response curve was fitted to 4 parameter Hill equation.

Testing of the compounds described in this invention was carried out using the SelectScreen™ platform from Invitrogen, Inc. (Carlsbad, Calif., USA) and the details of its performance are readily viewed via the web by linking to: http://www.invitrogen.com/downloads/SelectScrn_Brochure.pdf.

Briefly the approach is based on treating each specific kinase with a unique substrate and optical reporter system in the presence of ATP at 100 micromolar. In controls, the substrate is phosphorylated and a baseline optimal response is recorded.

Compounds were initially tested at 1000 nanomolar concentrations and the % inhibition of enzyme activity determined (E1000). The compounds were then re-tested by adding graded amounts of putative inhibitor which were added in 5 separate increments to generate a dose response curve. The latter is obtained by fitting to a 4 parameter Hill equation, a sigmoid saturation equation. The concentration which causes 50% enzyme inhibition (EC50) was then calculated from the dose response equation.

An effective level of inhibition in the low nanomolar range is considered to qualify the test compound as potential drug or targeting agent against the specific kinase that it has inhibited. The EC₅₀ value is, therefore, a measure of potency. Another important feature is specificity. It is considered a desirable property when claiming efficacy to determine how many kinases are inhibited by the same molecule. The fewer number inhibited points toward specificity; the greater to inhibitory promiscuity. For the compounds of this invention, the experimental condition were as follows.

The 2× FLT3/Tyr 02 peptide mixture was prepared in 50 mM HEPES pH 7.5, 0.01 BRIJ-35, 10 mM MgCl2, 1 mM EGTA. The final 10 uL kinase reaction consists of 0.6-76.0 ng FLT3 and 2 uM Tyr 02 peptide in 50 mM HEPES pH 7.5, 0.01% BRIJ-35, 10 mM Mg Cl2, 1 mM EGTA. After 1 hour kinase reaction incubation, 5 uL of a 1:64 dilution of development reagent A was added.

Values of EC50 and E1000 of representative compounds of the invention, measured in in vitro dose response assays against FLT3, are reproduced below.

| Structure | Name | EC50 | E1000 |
|---|---|---|---|
| 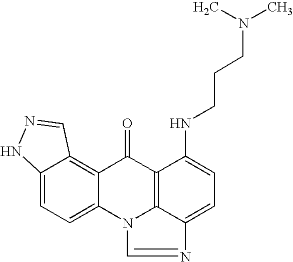 | XF-0123 | 9 | 97 |
| 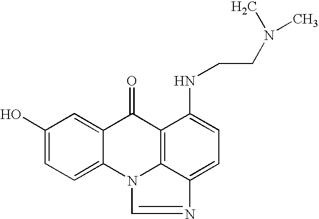 | XF-0025 | 25 | [symbol] |
| 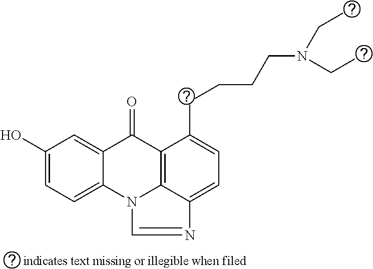 | XF-0226 | 23 | [symbol] |
| 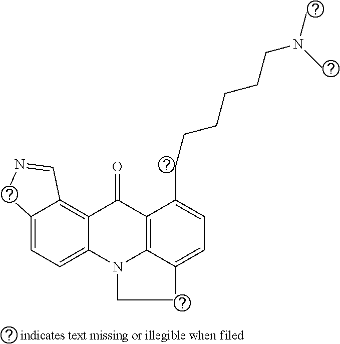 | XF-0124 | 16 | [symbol] |
| 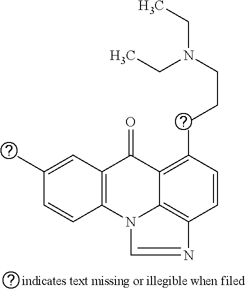 | XF-0302 | 25 | [symbol] |
| 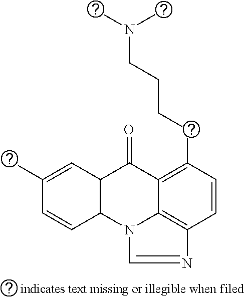 | XF-0318 | 19 | [symbol] |
| 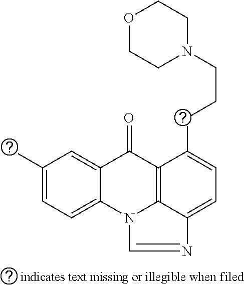 | XF-0322 | 23 | [symbol] |
| 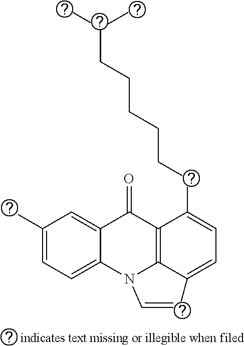 | XF-0207 | 23 | [symbol] |
| 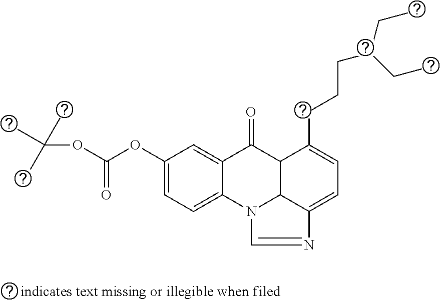 | XF-0332 | [symbol] | [symbol] |
| 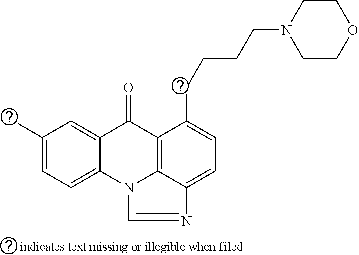 | XF-0113 | 50 | [symbol] |
| 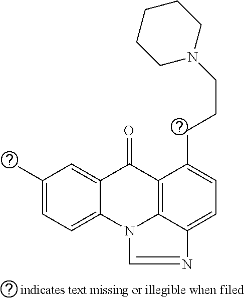 | XF-0321 | [symbol] | [symbol] |
| 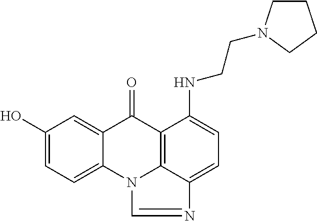 | XF-0241 | 50 | [symbol] |
| 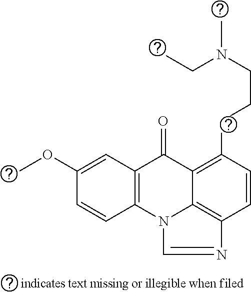 | XF-0015 | 128 | [symbol] |
| 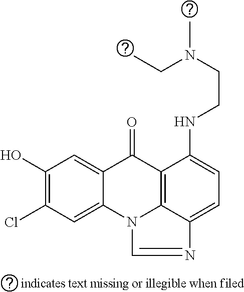 | XF-0020 | 140 | [symbol] |
| 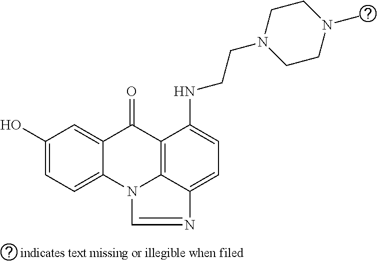 | XF-0043 | 77 | [symbol] |
| 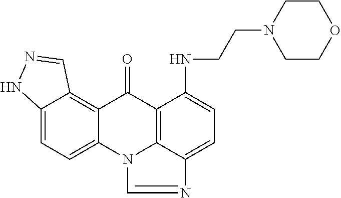 | XF-0122 | 51 | [symbol] |
| 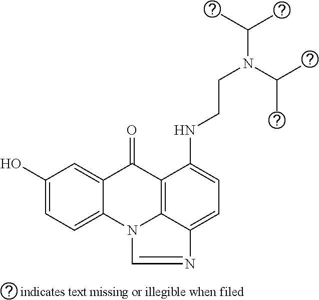 | XF-0121 | 32 | 87 |
| 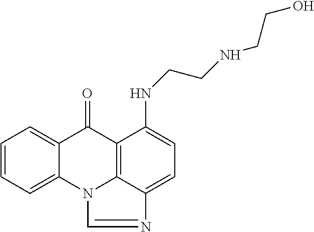 | XF-0328 | 135 | [symbol] |
| 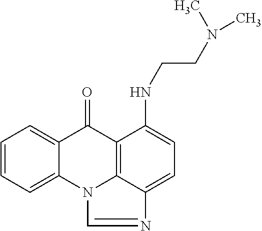 | XF-0308 | 133 | [symbol] |
| 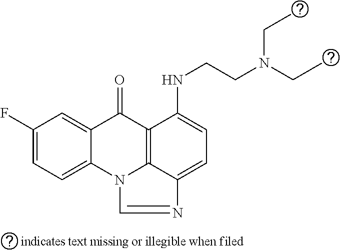 | XF-0348 | 196 | [symbol] |
| 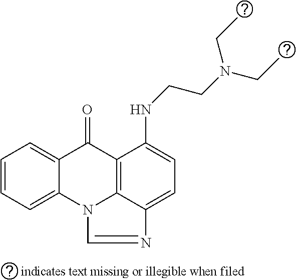 | XF-0227 | 224 | 78 |
| 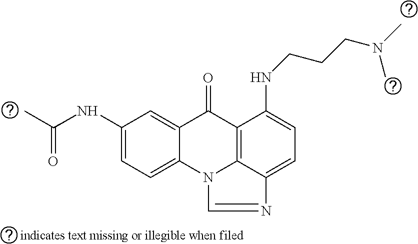 | XF-0111 | 277 | 74 |
| 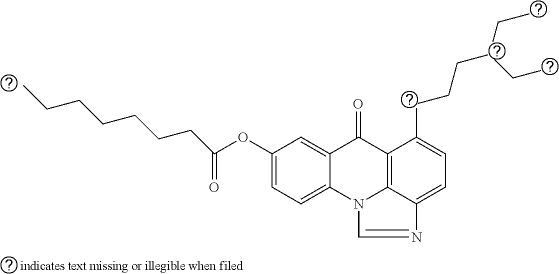 | XF-0132 | 277 | 74 |
| 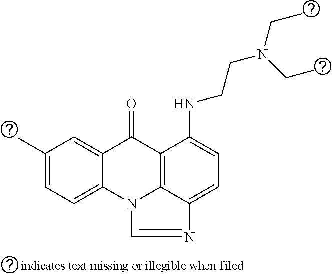 | XF-0136 | 342 | 70 |
| 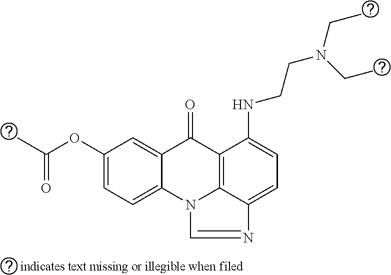 | XF-0129 | 471 | [symbol] |
| 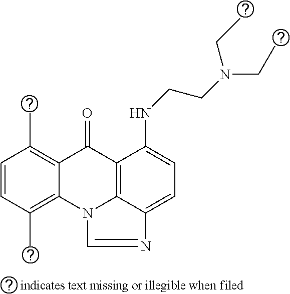 | XF-0330 | 496 | [symbol] |
| 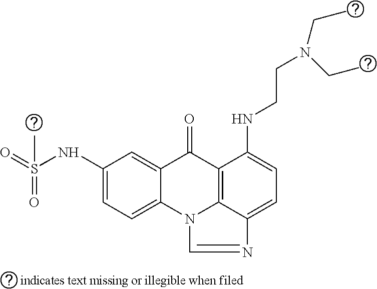 | XF-0144 | 297 | [symbol] |
| 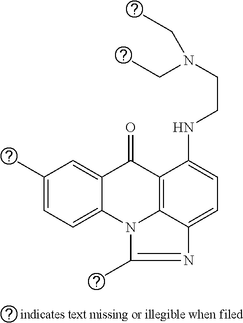 | XF-0006 | 795 | 52 |

[symbol]

indicates data missing or illegible when filed

While this invention has been particularly shown and described with references to preferred embodiments thereof, it will be understood by those skilled in the art that various changes in form and details may be made therein without departing from the scope of the invention encompassed by the appended claims.

1. A method of treating a FLT3-mediated condition in a patient in need thereof, comprising administering to the patient a therapeutically effective amount of a compound of formula (I) or a pharmaceutically acceptable salt thereof: 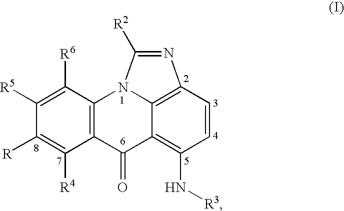
wherein R is:
R^{x}, or R and R⁴ or, alternatively, R and R⁵ taken together with their intervening carbon atoms form a 5, 6 or 7 member, optionally substituted, cycloalkyl or non-aromatic heterocycle containing one or two oxygens and optionally substituted with methyl or hydroxyl; or
R is a hydrolyzable group; or
R, alone or taken together with R⁴, or alternatively R⁵, and their intervening carbon atoms is a phenol isosteric group;
and further wherein:
R^{x} is —H, an optionally substituted alkyl, hydroxyl, alkoxy group, a halogen, or a group represented by the following structural formula: 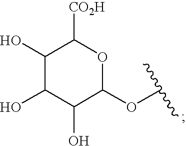
R² is —H, an optionally substituted C1-C10 alkyl or an optionally substituted aryl, optionally substituted aralkyl or optionally substituted heteroaryl;
R is —(CH₂)ₙ—NR^{a}R⁶, wherein n is an integer from 1 to 5, and R^{a} and R^{b}, each independently are hydrogen or an optionally substituted alkyl, or R^{a} and R^{b}, taken together with the nitrogen to which they are attached, form group R^{y},
wherein R^{y} is a heteroaryl or a non-aromatic heterocycle, each optionally substituted at one or more substitutable carbon atoms with methyl, hydroxyl, or methoxy, and optionally substituted at any substitutable ring nitrogen atom with C1-C4 alkyl or C1-C4 alkyl substituted with —NR^{c}R^{d}, wherein R^{c} and R^{d} are individually H, methyl or ethyl; and
R⁴, R⁵ and R⁶, are each independently —H, —OH, a halogen or optionally substituted C1-C6 alkoxy; or
R⁵ and R⁶ taken together with their intervening carbon atoms, form a 5, 6 or 7 member, optionally substituted cycloalkyl or optionally substituted non-aromatic heterocycle,
wherein the FLT3-mediated condition is one or more of axonal degeneration, acute transverse myelitis, amyotrophic lateral sclerosis, infantile spinal muscular atrophy, juvenile spinal muscular atrophy, Creutzfeldt-Jakob disease, subacute sclerosing panencephalitis, organ rejection, bone marrow transplant rejection, non-myeloablative bone marrow transplant rejection, ankylosing spondylitis, aplastic anemia, Behcet's disease, graft-versus-host disease, Graves' disease, autoimmune hemolytic anemia, Wegener's granulomatosis, hyper IgE syndrome, idiopathic thrombocytopenia purpura, and Myasthenia gravis.

2. The method of claim 1, wherein R is a hydrolysable group.

3. The method of claim 2, wherein R is selected from groups (II)-(VII): 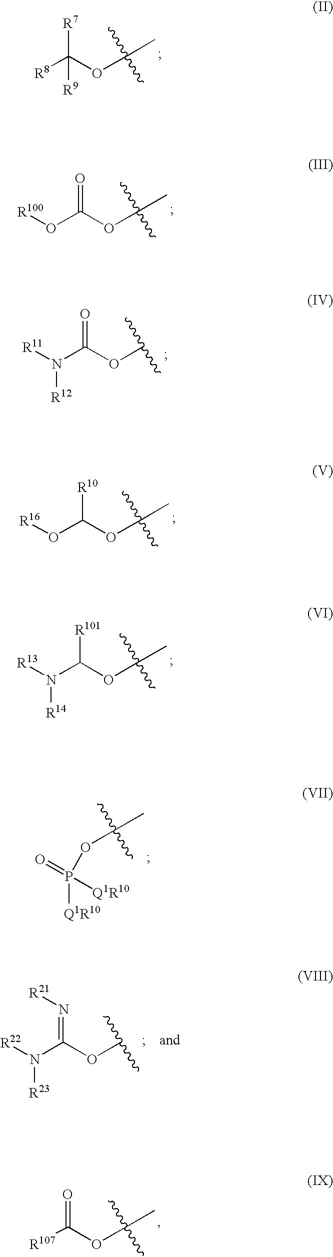
wherein
R⁷ and R⁸ are independently each H, optionally substituted C1-C6 alkyl, optionally substituted aryl or optionally substituted aralkyl;
R⁹ is carboxyl, carboxamide optionally N-substituted or N,N′-disubstituted with C1-C4 alkyl, C1-C6 alkanoyl, C1-C6 carbalkoxy, or optionally substituted aroyl;
R¹⁰ is H, optionally substituted C1-C6 alkyl or optionally substituted aryl or optionally substituted aralkyl;
R¹¹ and R¹² are independently each H, optionally substituted C1-C6 alkyl or, taken together with the atom to which they are attached, form an optionally substituted non-aromatic heterocycle;
R¹³ and R¹⁴ are each independently H, optionally substituted C1-C6 alkyl, optionally substituted C1-C6 alkanoyl, or optionally substituted aroyl, or, taken together with the atom to which they are attached, form an optionally substituted heteroaryl or non-aromatic optionally substituted heterocycle;
R¹⁶ is optionally substituted C1-C6 alkyl, optionally substituted aryl or optionally substituted aralkyl, optionally substituted C1-C6 alkanoyl, or optionally substituted aroyl;
R²¹ is optionally substituted C1-C10 alkyl, or an optionally substituted aryl or optionally substituted aralkyl or, R²¹ and R²² taken together with their intervening atoms form a 5-7 membered non-aromatic heterocycle;
R²² and R²³ are each independently —H, or a optionally substituted C1-C6 alkyl, provided that R²² and R²³ are not simultaneously hydrogens;
R¹⁰⁰ is optionally substituted C1-C6 alkyl or optionally substituted aryl or optionally substituted aralkyl;
R¹⁰¹ is H, optionally substituted C1-C6 alkyl or optionally substituted aryl or optionally substituted aralkyl;
R¹⁰⁷ is optionally substituted C1-C6 alkyl, optionally substituted aryl or aralkyl, or a non-aromatic heterocycle, optionally substituted at one or more substitutable carbon atoms with methyl, hydroxyl, or methoxy, and optionally N′-substituted at any substitutable nitrogen atom with C1-C4 alkyl or C1-C4 alkyl substituted with —NR^{c}R^{d}; and
Q¹ is O or NH.

4. The method of claim 3, wherein R^{y} is an optionally substituted heteroaryl or, in formula (I), n is an integer from 1 to 5 and R^{a} and R^{b}, each independently are hydrogen or an alkyl or R^{a} and R^{b}, taken together with the nitrogen to which they are attached form a 5-7 membered non-aromatic heterocycle optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl.

5. (canceled)

6. The method of claim 4, wherein in formula (II)
R⁷ and R⁸ are independently each H, C1-C6 alkyl, phenyl or benzyl, where the C1-C6 alkyl group, phenyl or benzyl represented by R⁷ and R⁸ are each optionally substituted with one or more hydroxyl, C1-C3 alkoxy, amino, alkylamino, halogen, haloalkyl or haloalkoxy groups; and
R⁹ is carboxyl, carboxamide optionally N-substituted or N,N′-disubstituted with a C1-C4 alkyl, C1-C4 alkanoyl, or C1-C4 carbalkoxy.

7. The method of claim 4, wherein:
R¹⁰ (formula (V) and, independently, in formula (VII)) is H or C1-C4 alkyl, phenyl, or benzyl, each optionally substituted with one or more hydroxyl, C1-C3 alkoxy, amino, alkylamino, halogen, haloalkyl or haloalkoxy groups;
R¹¹ and R¹² (formula (IV)) are independently each a H, methyl or ethyl or, taken together with the nitrogen atom to which they are attached form non-aromatic heterocycle, optionally substituted at one or more substitutable ring carbon atoms with methyl, hydroxyl, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl;
R¹⁶ (formula (V)) is a C1-C6 alkanoyl, optionally substituted with —OH, —SH, halogen, cyano, nitro, amino, —COOH, a C1-C3 alkyl, C1-C3 haloalkyl, C1-C3 alkoxy, C1-C3 haloalkoxy or C1-C3 alkyl sulfanyl, or —(CH₂)_{q}—C(O)OH, wherein and q are independently an integer from 1 to 6;
R¹⁰⁰ (formula (III)) is a C1-C4 alkyl;
R¹⁰⁷ (formula (IX)) is C1-C6 alkyl optionally substituted with —OH, —SH, halogen, cyano, nitro, amino, —COOH, a C1-C3 alkyl, C1-C3 haloalkyl, C1-C3 alkoxy, C1-C3 halo alkoxy or C1-C3 alkyl sulfanyl, or —(CH₂)_{q}—C(O)OH; and
the group of formula (VI) is represented by structural formulas (VIa) or (VIb): 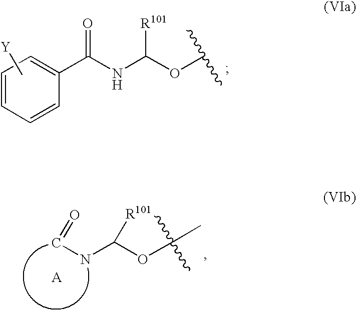
wherein:
Y is a halogen, —NO₂, —NH₂, —COOH, alkyl, C1-C3 carbalkoxy, C1-C3 alkoxy group, C1-C3 haloalkyl or C1-C3 haloalkoxy;
ring A is a 5-7 membered non-aromatic heterocycle optionally substituted at one or more substitutable ring carbon atoms with methyl, hydroxyl, oxy, or methoxy, and optionally substituted at any one or more ring nitrogen atoms with C1-C4 alkyl or C1-C4 alkyl substituted with —NR^{c}R^{d}, wherein R^{c} and R^{d} are individually H, methyl or ethyl; and
R¹⁰¹ is H or C1-C4 alkyl.

8. The method of claim 4, wherein in formula (VIII):
either R²¹ is optionally substituted C1-C10 alkyl, phenyl, or benzyl, each optionally substituted with a halogen, —NO₂, —NH₂, —COOH, alkyl, C1-C3 carbalkoxy, C1-C3 alkoxy group, C1-C3 haloalkyl or C1-C3 haloalkoxy, and R²² and R²³ are each independently —H, or a C1-C3 alkyl; or
R²¹ and R²², taken together with their intervening atoms, form a 5 or 6 membered non-aromatic heterocycle and R²³ is —H, or a C1-C3 alkyl.

9. The method of claim 8, wherein R² is —H, C1-C4 alkyl or C1-C4 haloalkyl, R⁴, R⁵ and R⁶ are each independently —H, —OH, C1-C4 alkyl or C1-C4 haloalkyl, or R⁵ and R⁶ taken together are methylenedioxy.

10. The method of claim 9, wherein:
either n is 2 or 3 and R^{a} and R^{b}, is each independently a hydrogen or a C1-C3 alkyl; or
R^{a} and R^{b}, taken together with the nitrogen to which they are attached, form group R^{y} selected form: 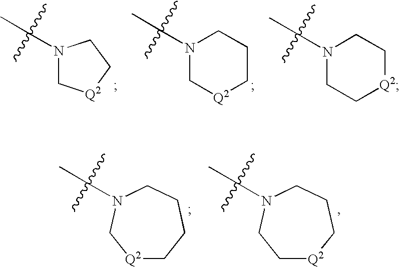
wherein Q² is S, O, CH₂, NH, or NR¹⁰², wherein R¹⁰² is methyl or ethyl.

11. The method of claim 10, wherein:
R⁷ and R⁸ (formula (II)) are each independently H, methyl or ethyl, and R⁹ is a C1-C4 alkanoyl;
each R¹⁰ (formula (V), and, independently, formula (VII)) is independently an H, or C1-C4 alkyl;
in formula (IV), NR¹¹R¹² is N-pyrrolidinyl, N-piperidinyl, N-morpholinyl, N-thiomorpholinyl or N-piperazinyl, optionally N′-substituted or N′,N′-disubstituted with C1-C4 alkyl or C1-C4 alkyl substituted with —NR^{c}R^{d}, wherein R^{c} and R^{d} are individually H, methyl or ethyl;
R¹⁰¹ (formula (VI)) is H, methyl or ethyl;
R¹⁶ (formula (V)) is a branched C3-C6 alkanoyl; and
R¹⁰⁷ (formula (IX)) is C1-C6 alkyl or C1-C6 carboxyalkyl.

12. The method of claim 11, wherein ring A is selected from:

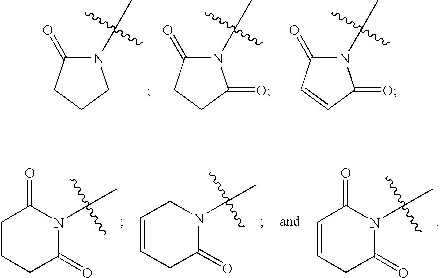

13. The method of claim 12, wherein R²—H, methyl or ethyl, and R⁶ is —H, —OH or methyl or ethyl.

14. The method of claim 1, wherein R alone or taken together with R⁴, or alternatively R⁵, and the intervening carbon atoms is the phenol isosteric group.

15. The method of claim 14, wherein the phenol isosteric group is selected from: 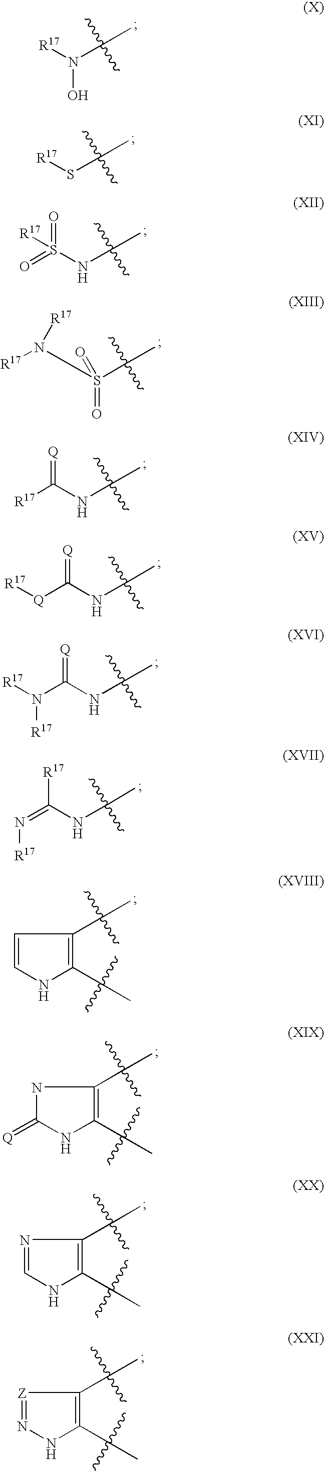 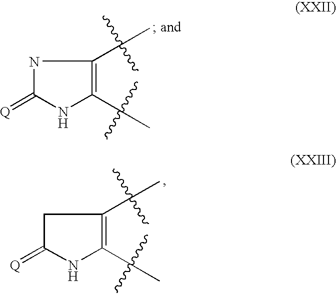
wherein
R¹⁷ for each occurrence, is H, optionally substituted C1-C6 alkyl, optionally substituted aryl or optionally substituted aralkyl or optionally substituted heteroaryl, C1-C6 alkoxyalkyl, optionally substituted aryloxy, optionally substituted aralkyloxy or optionally substituted heteroaryloxy;
Q is O or S; and
Z is CH or N.

16. The method of claim 15, wherein R^{y} is an optionally substituted heteroaryl or wherein in formula (I), n is an integer from 1 to 5, and R^{a} and R^{b,} each independently are hydrogen or an alkyl, or R^{a} and R^{b,} taken together with the nitrogen to which they are attached, form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d} wherein R^{c} and R^{d} are individually H, methyl or ethyl.

17. (canceled)

18. The method of claim 16, wherein R² is —H, C1-C4 alkyl or C1-C4 haloalkyl, and R⁵ and R⁶ are each independently —H, —OH, C1-C4 alkyl or C1-C4 haloalkyl, or taken together are methylenedioxy.

19. The method of claim 18, wherein R¹⁷ is H, optionally substituted C1-C6 alkyl, or C1-C6 alkoxyalkyl, or phenyl, benzyl, phenyloxy or benzyloxy each optionally substituted with halogen, —NO₂, —NH₂, —COOH, C1-C3 alkyl, C1-C3 carbalkoxy, C1-C3 a alkoxy group, C1-C3 haloalkyl or C1-C3 haloalkoxy.

20. The method of claim 19, wherein:
either n is 2 or 3 and R^{a} and R^{b}, is each independently a hydrogen or a C1-C3 alkyl; or
R^{a} and R^{b}, taken together with the nitrogen to which they are attached, form group R^{y} selected form a group consisting of 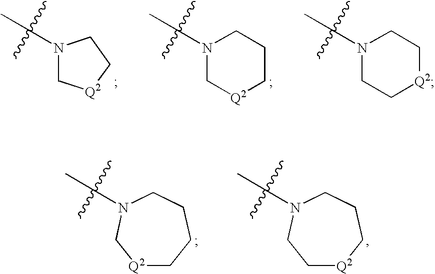
wherein Q² is Q² is S, O, CH₂, NH, or NR¹⁰², wherein R¹⁰² is methyl or ethyl.

21. (canceled)

22. The method of claim 20, wherein R¹⁷ is H, C1-C4 haloalkyl or phenyl optionally substituted with one or more halogen atoms or C1-C3 haloalkyls.

23. The method of claim 22, wherein R² is —H, methyl or ethyl; R⁶ is —H, —OH, methyl or ethyl; and R¹⁷ is H, trifluoromethyl or phenyl substituted with one or more trifluoromethyls.

24. (canceled)

25. The method of claim 1, wherein R is R^{x}, or R and R⁴ or, alternatively, R and R⁵ taken together with their intervening carbon atoms form a 5, 6 or 7 member, optionally substituted, cycloalkyl or non-aromatic heterocycle containing one or two oxygens and optionally substituted with methyl or hydroxyl.

26. The method of claim 23, wherein R^{y} is an optionally substituted heteroaryl or wherein in formula (I), n is an integer from 1 to 5, and R^{a} and R^{b,} each independently are hydrogen or an alkyl, or R^{a} and R^{b,} taken together with the nitrogen to which they are attached form a 5-7 member non-aromatic heterocycle, optionally substituted at one or more substitutable carbon atoms with methyl, hydroxyl, or methoxy, and optionally N′-substituted with C1-C4 alkyl or C1-C4 alkyl substituted with —NR^{c}R^{d} wherein R^{c} and R^{d} are individually H, methyl or ethyl.

27. (canceled)

28. The method of claim 26, wherein R² is an optionally substituted C1-C10 alkyl; and R is R^{x}.

29. (canceled)

30. The method of claim 28, wherein the compound is represented by structural formula (XXX):

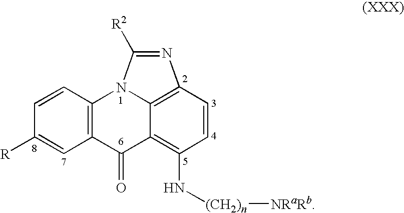

31. The method of claim 30, wherein R is —F, —OH or —OCH₃; n is 2 or 3; and R² is a —H or a C1-C4 alkyl.

32-33. (canceled)

34. The method of claim 31, wherein R^{a} and R^{b} are independently each a —H or an C1-C4 alkyl, or, taken together with the nitrogen to which they are attached, form a 5-7 membered nonaromatic heterocycle, and wherein the C1-C4 alkyl is optionally substituted with a hydroxyl, an amino, a C1-C4 N-alkyl-amino or a C1-C4 N,N-dialkylamino group.

35. The method of claim 34, wherein the substituents on R^{a} and R^{b} are independently hydroxyethyl, amino ethyl, N-alkylamino ethyl or N,N-dialkylaminoethyl.

36. The method of claim 35, wherein R is —F, —OH or —OCH₃, R^{a} and R^{b} are identical and are methyl or ethyl, or, taken together with the nitrogen atom to which they are attached, form a morpholino group; n is 2 or 3; R² is a hydrogen or a C1-C4 alkyl.

37. The method of claim 36, wherein the compound is represented by one of the following formulas:

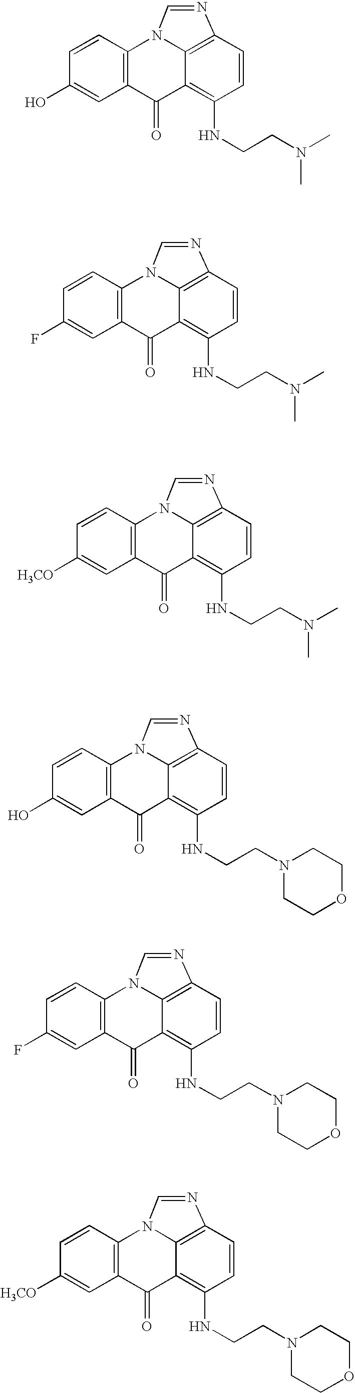

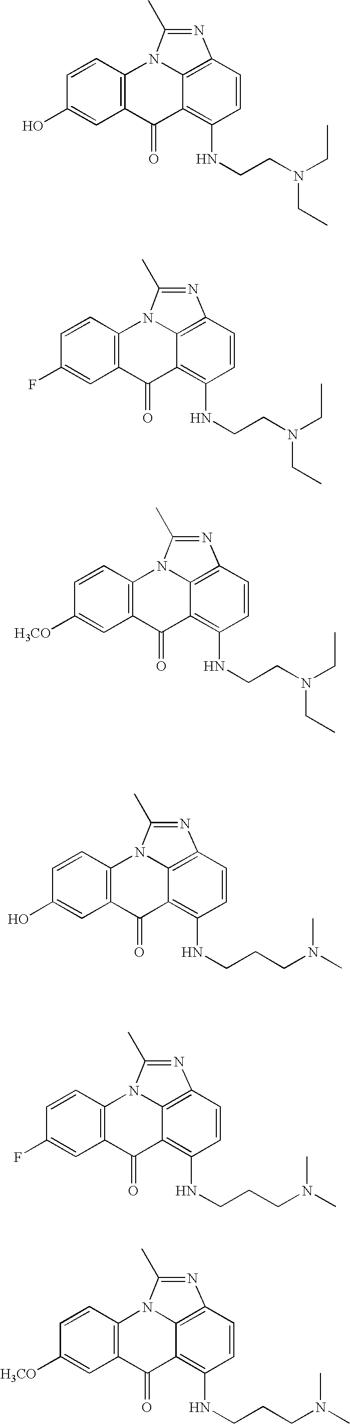

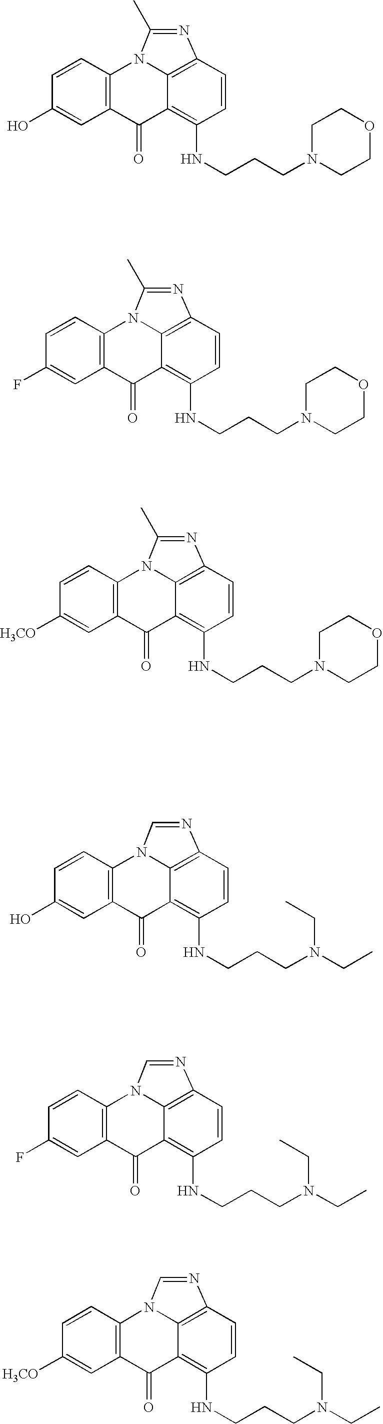

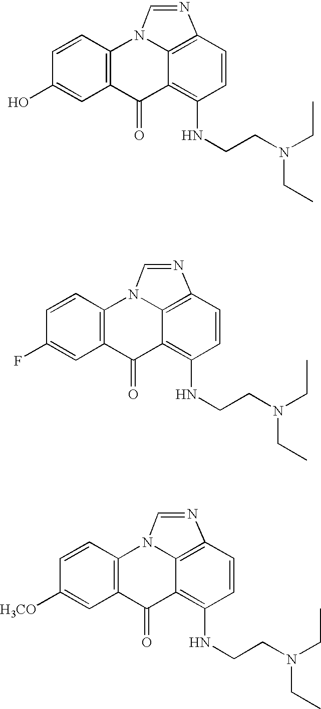

38. The method of claim 1, wherein one or more additional pharmaceutical agents is co-administered with a compound of formula (I).

39. A method of treating a FLT3-mediated condition in a patient in need thereof, comprising administering to the patient a therapeutically effective amount of a compound of formula (I) or a pharmaceutically acceptable salt thereof: 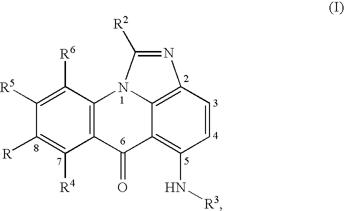
wherein R is:
R^{x}, or R and R⁴ or, alternatively, R and R⁵ taken together with their intervening carbon atoms form a 5, 6 or 7 member, optionally substituted, cycloalkyl or non-aromatic heterocycle containing one or two oxygens and optionally substituted with methyl or hydroxyl; or
R is a hydrolyzable group; or
R, alone or taken together with R⁴, or alternatively R⁵, and their intervening carbon atoms is a phenol isosteric group;
and further wherein:
R^{x} is —H, an optionally substituted alkyl, hydroxyl, alkoxy group, a halogen, or a group represented by the following structural formula: 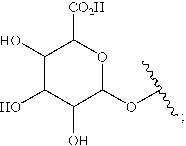
R² is —H, an optionally substituted C1-C10 alkyl or an optionally substituted aryl, optionally substituted aralkyl or optionally substituted heteroaryl;
R³ is —(CH₂)ₙ—NR^{a}R^{b}, wherein n is an integer from 1 to 5, and R^{a} and R^{b}, each independently are hydrogen or an optionally substituted alkyl, or R^{a} and R^{b}, taken together with the nitrogen to which they are attached, form group R^{y},
wherein R^{y} is a heteroaryl or a non-aromatic heterocycle, each optionally substituted at one or more substitutable carbon atoms with methyl, hydroxyl, or methoxy, and optionally substituted at any substitutable ring nitrogen atom with C1-C4 alkyl or C1-C4 alkyl substituted with —NR^{c}R^{d}, wherein R^{c} and R^{d} are individually H, methyl or ethyl; and
R⁴, R⁵ and R⁶, are each independently —H, —OH, a halogen or optionally substituted C1-C6 alkoxy; or
R⁵ and R⁶ taken together with their intervening carbon atoms, form a 5, 6 or 7 member, optionally substituted cycloalkyl or optionally substituted non-aromatic heterocycle,
wherein the compound of formula (I) reduces the activity of FLT3 with not more than 1% cross-inhibition of any other tyrosine kinase receptor.
